(19) United States
(12) Patent Application Publication
(10) Pub. No.: US 2002/0137074 A1
(43) Pub. Date: Sep. 26, 2002

(54) Selectivity of nucleic acid diagnostic and microarray technologies by control of interfacial nucleic acid film chemistry

(76) Inventors: Paul A.E. Piunno, Mississauga (CA); James H. Watterson, Mississauga (CA); Christopher C. Wust, Mississauga (CA); Ulrich J. Krull, Mississauga (CA)

Correspondence Address: GREENLEE WINNER AND SULLIVAN P C, 5370 MANHATTAN CIRCLE, SUITE 201, BOULDER, CO, 80303, US

(21) Appl. No.: 09/993,303

(22) Filed: Nov. 21, 2001

Related U.S. Application Data
(60) Provisional application No. 60/252,643, filed on Nov. 21, 2000.

(51) Int. Cl. C12Q 1/68; C12M 1/34

(52) U.S. Cl. 435/6; 435/287.2

(57) ABSTRACT

The invention provides methods for conducting hybridizations having increased selectivity of hybridization using substrates upon which probe nucleic acids are immobilized. The methods of this invention can be used to increase selectivity in nucleic acid diagnostic devices, such as biosensors and microarrays. The invention provides increased selectivity through control of the substrate surface chemistry and in particular, through control of the density of nucleic acids and other oligomers immobilized on a surface. The invention provides improved signal to noise in hybridization assays via enhanced differences in signal magnitude generated for fully matched target nucleic acid compared to partially matched target nucleic acid prior to signal processing. Specifically, invention provides methods for using substrates having medium-high to high immobilization densities to achieve higher hybridization The methods and substrates of this invention are particularly well-suited to assays for genetic targets in samples that contain genetic species that are very similar in nucleic acid sequence to the genetic target. The methods and substrates of this invention are also well-suite for single nucleotide polymorphism (SNP) analysis.

### CROSS-REFERENCE TO RELATED APPLICATIONS

This application takes priority under 35 U.S.C. §119(e) to U.S. provisional patent application No. 60/252,643, filed Nov. 21, 2000, which is incorporated by reference in its entirety herein.

### FIELD OF THE INVENTION

The invention relates to methods of increasing selectivity of nucleic acid diagnostic devices, such as biosensors and microarrays.

### BACKGROUND OF THE INVENTION

The immobilization of biomolecules to solid surfaces is widely used in the preparation of analytical sensors. Applications include immunosensor techniques [1,2,3], which tend to rely on protein binding as the means of molecular “recognition”, as well as those which make use of nucleic acid hybridization [4,5,6,7,8,9] as the basis for selective recognition. The use of immobilized nucleic acids to provide for selective binding interactions is attractive since the selectivity of nucleic acid binding interactions can be quite high and the advent of polymerase chain reaction and solid phase nucleic acid synthesis has allowed for relatively simple nucleic acid preparation and immobilization.

The utility of immobilized selective molecular recognition elements is dependent upon the retention of selective binding capacity after the immobilization process is complete. The binding capacity is dependent upon the structure of the immobilized molecules in their local environments, which can be significantly different from those experienced in bulk solution. The density of immobilization of single-stranded DNA (ssDNA) onto the surface of a solid substrate affects the charge density at the surface, and the extent to which the immobilized oligomers interact with the surface of the solid substrate and with neighbouring nucleic acid oligomers. The density of immobilization thus affects the extent of hybridization, as well as the orientation of the immobilized ssDNA, and therefore affects the kinetics of hybridization [10]. Clearly, the control of selectivity of binding and the dynamic range that can be achieved by control of the concentration of oligonucleotide sequences at an interface is complex.

The binding capacity of immobilized oligonucleotides is dependent in part upon the structure and orientation of the oligonucleotides in the interfacial environment, which is dictated at least in part by chemical nature of the solid substrate. Control and elucidation of the orientation and packing structure of nucleic acids immobilized on gold and polystyrene surfaces has been attempted [11,12,13]. It was suggested that the alignment of immobilized oligonucleotides with respect to the substrate surface can be controlled by selection of oligonucleotide immobilization density, as well as through control of the chemical environment at the surface. For example, Tarlov et al. [11] reported that adsorptive interactions of oligonucleotides immobilized by sulfur-gold interactions on a gold surface were reduced by blocking unreacted surface sites with mercaptohexanol. The reduction in oligonucleotide adsorption to gold resulted in extension of the immobilized oligonucleotides away from the substrate surface. The extent of hybridization was found to be affected by the packing density of immobilized oligonucleotides, with hybridization being inhibited at higher packing densities where steric hindrance and electrostatic repulsion were thought to reduce the stability of hybrids that could form. Alternatively, Fotin et al. [14] reported a method for large-scale parallel thermodynamic analysis of oligonucleotide hybridization using oligonucleotides immobilized in an array of polyacrylamide gel pads, each of dimension 100×100×20 μm. This method of immobilization was claimed to be well-suited for large scale thermodynamic analysis of oligonucleotide hybridization because the local environment experienced by the immobilized oligonucleotides afforded by the polyacrylamide gel more closely resembled that of a homogeneous liquid phase than that of the heterogeneous solid-liquid interface obtained when DNA is immobilized onto gold, silica, or polystyrene. Consequently, the method was presented as a means to estimate thermodynamic properties of oligonucleotide hybrids in solution based on the properties observed in experiments done within the gel-pad environment.

The binding capacity of immobilized nucleic acids is also dependent upon the extent to which neighbouring oligonucleotides can interact with each other. Shchepinov et al. [15] reported on the effects of the length of the linker molecule separating the immobilized oligonucleotide from the solid substrate surface on the extent of hybridization. They reported the observation of an optimal linker length of approximately 40 atoms, beyond which reductions in hybridization efficiency were attributed to increased interactions between neighbouring oligonucleotides that imparted steric hindrance to hybridization. It has also been suggested that the density of immobilization of oligonucleotides on polystyrene latex particles effects the orientation of the immobilized strands relative to the surface. Winnik et al. [14] used fluorescence resonance energy transfer (FRET) to examine the proximity of fluorescein-labelled oligonucleotides (donor) to immobilized tetramethylrhodamine moieties (acceptor), and thereby give a relative measure of immobilized oligonucleotide conformation relative to the substrate surface under a variety of experimental conditions. In addition to reporting that solution conditions such as pH and ionic strength affect the conformation of immobilized oligonucleotides, they reported that increasing the density of immobilized oligonucleotides also reduced the extent of energy transfer between the fluorescein and tetramethylrhodamine moieties, suggesting that as oligonucleotide packing density increased, the immobilized strands extended further away from the substrate surface due to electrostatic repulsion between neighbouring polyanionic strands.

The development of microarray technologies has stemmed from the desire to examine very large numbers of nucleic acid probe sequences simultaneously, in an effort to obtain information about genetic mutations, gene expression or nucleic acid sequences. Microarray technologies are intimately connected with the Human Genome Project, which has development of rapid methods of nucleic acid sequencing and genome analysis as key objectives [16]. Genome mapping and elucidation of sequence-function relationships will provide a wealth of knowledge about all stages of human development and aging, as well as, the onset of and predisposition to disease [17].

Oligonucleotide arrays have been developed as a hybridization “template” where a target sequence can be examined for its ability to hybridize to large numbers of different immobilized oligonucleotide sequences. These systems have been the focus of much research and have been reviewed [18,19]. One such system has been developed at Affymetrix, Inc. [20] that makes use of photolithographic techniques to direct spatially addressed synthesis of polynucleotides [21]. Arrays are synthesized on solid glass supports that have been coated with amino-terminated linkers to which photolabile nitroveratryloxycarbonyl (NVOC) groups have been added. Photo-deprotection of selected areas is achieved by illuminating those target areas through a photolithographic mask. Subsequent exposure of the entire chip to amino acid or nucleotide reagents results in reaction only at the selectively deprotected sites. Thus, site-specific synthesis is achieved through repetition of these steps and use of the appropriate photolithographic masks. Hybridization of these probe sequences with fluorescently-labelled target polynucleotidescan then be done and the array can be scanned by means of scanning fluorescence microscopy. The fluorescence patterns are then analyzed by an algorithm that determines the extent of mismatch content, identifies polymorphisms and can provide some general sequencing information [22]. Selectivity is afforded in this system by low stringency washes to rinse away non-selectively adsorbed materials. Subsequent analysis of relative binding signals from array elements determines where base-pair mismatches may exist. This method then relies on conventional chemical methods to maximize stringency, and automated pattern recognition processing is used to discriminate between fully complementary and partially complementary binding.

Another oligonucleotide array system has been developed by Nanogen Inc. [23]. An array of platinum microelectrodes was fabricated on silicon wafers using photolithography. One example of such an array device consisted of 25 microelectrodes, 80 μm in diameter, and four microelectrodes, 160 μm in diameter occupying outer corner positions of the array. Each electrode was covered with an agarose permeation layer that permitted ion transport to and from the electrode surface while serving as a site for attachment of probe oligonucleotides. The permeation layer also served as a “spacer” layer that acted to sufficiently separate the probe oligonucleotides from the electrode surfaces to protect the DNA from damaging redox reaction sites. Each electrode in the array was independently connected to an external power source. A continuously adjustable potential or current could be directed to each electrode via computer-controlled switching. This allowed each electrode to be maintained at a positive, negative or neutral bias with respect to the power supply. In one example, immobilization of probe DNA was achieved by incorporating streptavidin into the agarose permeation layer and directing biotinylated oligonucleotides to the layer by applying a positive potential at the target electrode sites. The extent of immobilization using positive, negative and neutral biases was examined by using fluorescently labelled oligonucleotides in the immobilization. It was observed that significant immobilization occurred only at those sites that were at a positive applied potential. This immobilization was also observed to be irreversible by switching the potential of the electrode and applying a strong negative potential. Hybridization of labelled target DNA was then carried out using electric field control as described above. It was found that hybridization to complementary DNA immobilized at electrodes with a positive applied potential occurred 25 times faster than hybridization at neutral electrodes. Reversal of the electric field was then used to examine the ability of the system to discriminate between hybrids of complete complementarity and those that contained single base-pair mismatches. It was observed that electrodes where hybrids were completely complementary retained 70% of the original fluorescent signal, whereas electrodes where hybrids contained single base-pair mismatches retained only 13% of the original fluorescent signal (i.e., a selectivity ratio of only about 5.4). This ability to discriminate between fully complementary hybrids and those containing single base-pair mismatches was observed with hybrids of different length and G-C content, and was found to occur quite rapidly, with full signal achieved in 15 seconds or less. Overall, this system is significant since it shows that controlling the electrochemical environment of the hybrids affects the selectivity of hybridization in an assay.

Devices such as standard nucleic acid microarrays or gene chips, require complicated data processing algorithms and the use of a high level of sample redundancy (i.e. many of the same types of array elements for statistically significant data interpretation and avoidance of anomalies) to provide semi-quantitative analysis of polymorphisms or levels of mismatch between the target sequence and that immobilised on the device surface.

There remains a need in the art to improve control of surface chemistry in order to obtain suitable hybridization selectivity.

### SUMMARY OF THE INVENTION

The invention relates to methods for increasing the selectivity of hybridization of probe nucleic acids immobilized on substrate surfaces to other nucleic acids. The methods of this invention can be used to increase selectivity in nucleic acid diagnostic devices, such as biosensors and microarrays, which detect the presence of nucleic acid in a test sample through detection of hybridization between the immobilized probe nucleic acid and nucleic acids in a test sample. The invention provides increased selectivity through control of the substrate surface chemistry and in particular, through control of the density of nucleic acids and other oligomers immobilised on a surface. The invention provides improved signal to noise in hybridization assays via enhanced differences in signal magnitude generated for fully matched target nucleic acid as opposed to partially matched target nucleic acid prior to signal processing. This makes the task of signal processing less onerous, time consuming and complex.

Furthermore, control of the substrate surface chemistry can be used to adjust the effective duplex melting temperature (Tₘ) so that combinations or arrays of immobilised nucleic acid films (a layer of immobilized oligomers) in a system can be made to be of similar Tₘ, regardless of immobilized nucleotide length and sequence. This will allow for simultaneous analysis of many interfacial hybridisations, facilitating enhanced high throughput screening capacity. The properties of immobilized nucleic acids described in this invention are applicable to many different devices using various types of nucleic acid immobilization strategies that will be apparent to one of ordinary skill in the art.

In specific embodiments the invention provides substrates for carrying out nucleic acid hybridization reactions in which a plurality of first nucleic acids are immobilized on the substrate surface alone or in combination with other oligomers at medium-high to high immobilization density.

The specific embodiments the invention provides methods for using substrates having such medium-high to high immobilization densities to achieve higher hybridization selectivity between fully complementary nucleic acids and those that have one or more mismatches in sequence. The invention includes improved methods for detecting target nucleic acids and for isolating target nucleic acids. More specifically the invention related to improved methods for detecting genetic targets, such as microorganisms and genes. The methods of this invention are particularly well-suited to assays for genetic targets in samples that contain genetic species that are very similar in nucleic acid sequence to the genetic target.

### BRIEF DESCRIPTION OF THE DRAWINGS

The invention is further illustrated and described in the following figures:

FIG. 1. Reaction Scheme for the functionalisation of fused silica substrates with DMT-HEG linkers;

FIG. 2. AE-HPLC Chromatogram of low-density preparation of immobilized oligonucleotides.

FIG. 3. AE-HPLC Chromatogram of medium-density preparation of immobilized oligonucleotides.

FIG. 4. AE-HPLC Chromatogram of high-density preparation of immobilized oligonucleotides.

FIG. 5. Synthetic Scheme for the preparation of DMB-HEG linker.

FIG. 6. Synthetic Scheme for the preparation of the Ethylene glycol phosphoramidite synthon.

FIG. 7. AE-HPLC Chromatogram of high-density preparation of immobilised oligonucleotides by use of the methods recited in Example 4.

FIG. 8. AE-HPLC Chromatogram of high-density preparation of immobilised films composed of a mixture of oligonucleotide-linker conjugates and ethyleneglycolphosphate-based oligomer linker conjugates by use of the methods recited in Example 4

FIG. 9. (a) Uncorrected fluorescence thermal denaturation profile from an optical fibre that was functionalised with dT₂₀ of low oligonucleotide packing density (372 Å centre-to-centre separation distance) and reacted with 10⁻⁷ M dA₂₀-5′-Fluorescein in 1.0×PBS buffer. Fitted curves are shown for (b) upper and (c) lower baseline.

FIG. 10. Baseline corrected and normalised thermal denaturation profiles from optical fibres that were functionalised with dT₂₀ at (a) low oligonucleotide packing density (372 Å centre-to-centre separation distance) reacted with solutions of 10⁻⁷ M dA₂₀-5′-Fluorescein in (i) 0.1×PBS buffer, (ii) 0.5×PBS buffer, and (iii) 1.0×PBS buffer. Raw data for profile (a) is shown in FIG. 9.

FIG. 11. Uncorrected thermal denaturation profiles: (a) from an optical fibre that was functionalised with dT₂₀ of high oligonucleotide packing density (20 Å mean centre-to-centre separation distance) and reacted with 10⁻⁷ M dA₂₀-5′-Fluorescein and 10⁻⁷ M dA₁₀GA₉-5′-Fluorescein in 0.5×PBS buffer and (b) from an optical fibre that was functionalised with immobilised dT₂₀ and Ethyleneglycolphosphate-based oligomers in a 1:20 ratio of high oligonucleotide packing density (50 Å mean centre-to-centre separation distance) and reacted with 10⁻⁷ M dA₂₀-5′-Fluorescein and 10⁻⁷ M dA₁₀GA₉-5′-Fluorescein in 0.5×PBS buffer.

FIG. 12. Normalised thermal denaturation profiles from an optical fibre that was functionalised with dT₂₀ of high oligonucleotide packing density (20 Å mean centre-to-centre separation distance) and reacted with 10⁻⁷ M dA₂₀-5′-Fluorescein and 10⁻⁷ M dA₁₀GA₉-5′-Fluorescein in (a) 0.1×PBS, (b) 0.5×PBS and (c) 1.0×PBS buffer and from an optical fibre that was functionalised with dT₂₀ and Ethyleneglycolphosphate-based oligomers in a 1:20 ratio of high oligonucleotide packing density (50 Å mean centre-to-centre separation distance) and reacted with 10⁻⁷ M dA₂₀-5′-Fluorescein and 10⁻⁷ M dA₁₀GA₉-5′-Fluorescein in (d) 0.1×PBS, (e) 0.5×PBS and (f) 1.0×PBS buffer. Comparison of the relative sensitivity to temperature for each type of sensor as a function of buffer ionic strength.

FIG. 13: Scheme for exemplary preparation of mixed immobilized layers of nucleic acids and other oligomers

### DETAILED DESCRIPTION OF THE INVENTION

Definitions:

“The length of an immobilized oligomer” is the physical length of the oligomers plus the length of any linker by which the oligomer is tethered to the substrate surface. In cases in which the oligomer is branched, the physical length of the oligomer is defined as the length of the longest chain of the oligomer. In cases in which different oligomers are immobilized, an “average length of the immobilized oligomers” is calculated using the length of the different immobilized oligomers and the number density of different oligomers immobilized.

“Low immobilization density” refers to the density of oligomers immobilized on a substrate where immobilized oligomers, including nucleic acids, are sufficiently separated such that no physical interactions can occur between neighbouring oligomers. Qualitative definitions of immobilization density depend not only on absolute number density of immobilized nucleic acid and any other co-immobilized oligomers, but also on the average dimensions of the immobilized nucleic acid and any other immobilized oligomers. Consequently, low immobilization density is represented by the case where the ratio (rₛ) of the mean center-to-center separation distance between neighbouring oligomers (nucleic acids or other oligomers) to the average length of the oligomers is significantly greater than two. It will be appreciated by those of ordinary skill in the art that the length of an immobilized oligomer calculated based on the structure of the oligomer and any linker to which it may be attached is an estimate of the space on the substrate surface that can be occupied by the immobilized oligomer. Immobilized oligomers may occupy a larger area than expected based on their length due to the effect of molecular shape or orientation, the effect of extended solvent structure (e.g., hydration), the effect of the electrostatic field of the oligomer and the like.

“Moderate or medium density” refers to the density of oligomers immobilized on a substrate where interactions between neighbouring oligomers may just be physically possible and is represented by the case where rₛ, as defined above, approaches but is greater than 2.

“Medium-high immobilization density” refers to the density of oligomers immobilized on a substrate where significant interaction between neighbouring oligomers is likely and is represented by the case where the ratio (rₛ) as defined above is greater than 1.7 and less than or equal to 2.

“High immobilization density” refers to the case where the density of oligomers immobilized on a substrate where significant interaction between neighbouring oligomers is probable and is represented by the case where the ratio (rₛ) as defined above is less than or equal to 1.7.

“High ionic strength” refers to a solution with an ionic strength of at least 0.3 M, and alternatively of at least 0.5M.

“Inversion effect” or “Tₘ inversion effect” refers to the observation that a difference in Tₘ between

(i) a fully-matched complex immobilized to a substrate, the complex comprising a first nucleic acid and a second nucleic acid where the sequence of the first and second nucleic acids are complements; and

(ii) a mismatch complex immobilized to a substrate, the complex comprising the first nucleic acid and a second nucleic acid having a single nucleotide mismatch;

when the immobilization density of oligomers on the substrate is medium-high or high density does not decrease and preferably increases compared to the difference in Tₘ between the aforementioned complexes when the immobilization density of oligomers on the substrate is low or medium density. The inversion effect permits maintenance of selectivity or, preferably, enhancement of selectivity at medium-high or high immobilization density compared to lower immobilization densities and in other environments where the inversion is not observed (e.g., bulk solution). A specific Tₘ inversion effect is observed when ionic strength of the sample is increased.

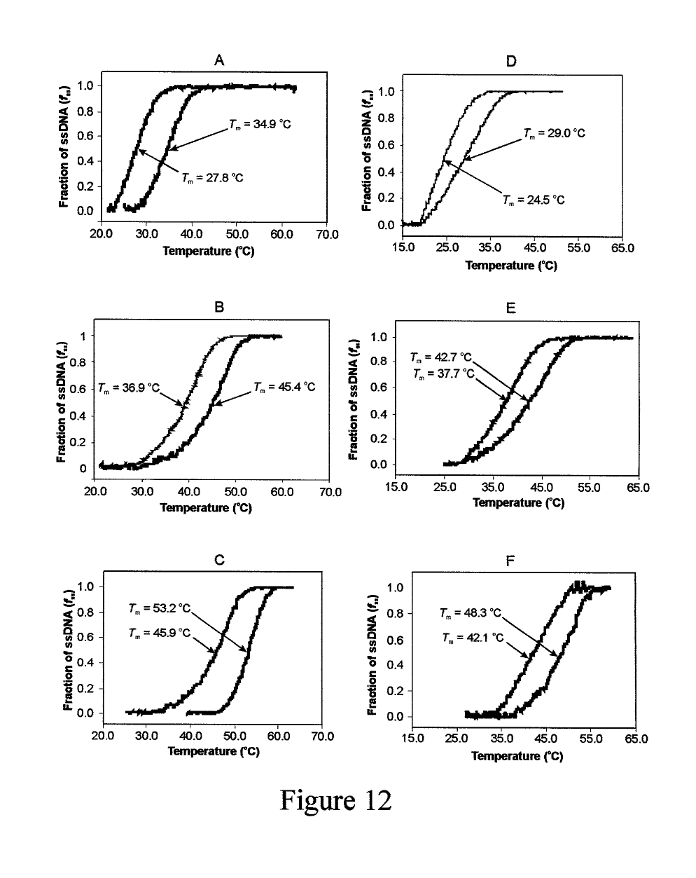

“Enhancement of temperature sensitivity” refers to an increase in the slope of thermal denaturation profiles. One application of this concept is to design the sensitivity of the experiment so that the operating temperature(s) for a sensor device (or the temperature(s) at which hybridization is performed) can be selected so that signal from one base pair mismatches is significantly smaller (preferably 10 fold lower) than signal from the fully complementary material. In a more preferable embodiment, operating temperature(s) can be selected where essentially all signal comes from fully-complementary material. Hybrizations performed with substrates having medium-high to high immobilization densities of nucleic acids, alone or in combination with other oligomers, can exhibit enhanced temperature sensitivity such that operating temperatures can be selected from thermal denaturation profiles such as those illustrated in FIG. 12 in which hybridization selectivities of 10 or more can be obtained. This level of selectivity enhancement has been observed in the hybridisation of nucleic acids of about 20 nucleotides and analogous nucleic acids containing a single base pair mismatch. Selectivity will increase over that observed for 20-mers for systems of shorter nucleic acids given the proportionally larger contribution to overall hybrid destabilization brought on by the single base pair mismatch. Dependent upon the length of the nucleic acids hybridized selectivities of 10, 20, 50, 100 or more can be achieved employing the methods and substrates of this invention between pairs of fully complementary nucleic acids and pairs of nucleic acids having a single base pair mismatch. Of course selectivities will be even greater between pairs of fully complementary nucleic acids and pairs of nucleic acids having more than one base pair mismatch.

The “middle” of a nucleic acid refers to the numerical middle nucleotide (if there is an odd number of nucleotides in a strand) or the numerical middle nucleotide pair (if there are an even number of nucleotides in a strand). Nucleic acid proximate to the middle of a molecule will preferably be within 10, 5, 2 or 1 nucleotide(s) of the middle of the nucleic acid. The middle of the hybridized portion would be the numerical middle of only that portion of the nucleic acid that is hybridized.

“Nucleic acid” includes DNA and RNA, whether single or double stranded. The term is also intended to include a strand that is a mixture of nucleic acids and nucleic acid analogs and/or nucleotide analogs, or that is made entirely of nucleic acid analogs and/or nucleotide analogs and that may be conjugated to a linker molecule.

“Nucleic acid analogue” refers to modified nucleic acids or species unrelated to nucleic acids that are capable of providing selective binding to nucleic acids or other nucleic acid analogues. As used herein, the term “nucleotide analogues” includes nucleic acids where the internucleotide phosphodiester bond of DNA or RNA is modified to enhance bio-stability of the oligomer and “tune” the selectivity/specificity for target molecules (Ulhmann, et al., 1990, Angew. Chem. Int. Ed. Eng., 90: 543; Goodchild, 1990, J. Bioconjugate Chem., 1: 165; Englisch et al., 1991, Angew, Chem. Int. Ed. Eng., 30: 613). Such modifications may include and are not limited to phosphorothioates, phosphorodithioates, phosphotriesters, phosphoramidates or methylphosphonates. The 2′-O-methyl, allyl and 2′-deoxy-2′-fluoro RNA analogs, when incorporated into an oligomer show increased biostability and stabilization of the RNA/DNA duplex (Lesnik et al, 1993, Biochemistry, 32: 7832). As used herein, the term “nucleic acid analogues” also include alpha anomers (α-DNA), L-DNA (mirror image DNA), 2′-5′ linked RNA, branched DNA/RNA or chimeras of natural DNA or RNA and the above-modified nucleic acids. For the purposes of the present invention, any nucleic acid containing a “nucleotide analogue” shall be considered as a nucleic acid analogue. Backbone replaced nucleic acid analogues can also be adapted to for use as immobilised selective moieties of the present invention. For purposes of the present invention, the peptide nucleic acids (PNAs) (Nielsen et al., 1993, Anti-Cancer Drug Design, 8: 53; Engels et al., 1992, Angew, Chem. Int. Ed. Eng., 31: 1008) and carbamate-bridged morpholino-type oligonucleotide analogs (Burger, D. R., 1993, J. Clinical Immunoassay, 16: 224; Uhlmann, et al., 1993, Methods in Molecular Biology, 20,. “Protocols for Oligonucleotides and Analogs,” ed. Sudhir Agarwal, Humana Press, NJ, U.S.A., pp. 335-389) are also embraced by the term “nucleic acid analogues”. Both exhibit sequence-specific binding to DNA with the resulting duplexes being more thermally stable than the natural DNA/DNA duplex. Other backbone-replaced nucleic acids are well known to those skilled in the art and can also be used in the present invention (See e.g., Uhlmann et al., 1993, Methods in Molecular Biology, 20, “Protocols for Oligonucleotides and Analogs,” ed. Sudhir Agrawal, Humana Press, NJ, U.S.A., pp. 335).

A genetic marker nucleic acid is the complement of a nucleic acid the presence of which in a test sample indicates the presence of a genetic target, such as a microorganism or a specific gene. In some cases a single genetic marker nucleic acid can be used to detect the presence of a genetic target. In other cases more than one genetic marker nucleic acid will be necessary to detect the presence of a genetic target.

“Oligomer” refers to a polymer that consists of two or more monomers that are not necessarily identical. Oligomers include, without limitation, nucleic acids (which include nucleic acid analogs as defined above), oligoelectrolytes, hydrocarbon based compounds, dendrimers, nucleic acid analogues, polypeptides, oligopeptides, polyethers, oligoethers any or all of which may be immobilized to a substrate. Oligomers an be immobilized to a substrate surface directly or via a linker molecule.

“Selectivity” or “hybridization selectivity” is the ratio of the amount of hybridization (i.e., number of second nucleic acids hybridized) of fully complementary hybrids to partially complementary hybrids, based on the relative thermodynamic stability of the two complexes. For the purpose of this definition it is presumed that this ratio is reflected as an ensemble average of individual molecular binding events. Selectivity is typically expressed as the ratio of the amount of hybridization of fully complementary hybrids to hybrids having one base pair mismatches in sequence. Selectivity is a function of many variables, including, but not limited to,: temperature, ionic strength, pH, immobilization density, nucleic acid length, the chemical nature of the substrate surface and the presence of polyelectrolytes and/or other oligomers immobilized on the substrate or otherwise associated with the immobilised film. Selectivity values that can be obtained using the methods and substrates of this invention will generally increase for systems of shorter nucleic acids given the proportionally larger contribution to overall hybrid destabilization brought on by single base pair mismatches.

“Selectively hybridize” refers to hybridization under conditions where there would be a difference in Tₘ of at least 5, 6, 7, 8, 9, 10, 11 or 12 degrees Celsius between

(i) a fully-matched complex immobilized to a substrate, the complex comprising a first nucleic acid and a second nucleic acid where the sequences of the first and the second nucleic acid are complements; and

(ii) a mismatch complex immobilized to a substrate, the complex comprising the first nucleic acid and a second nucleic acid having a single nucleotide mismatch from the first nucleic acid.

The present invention is directed generally to hybridization methods exhibiting enhanced selectivity. Such methods can be applied to the identification and analysis of target nucleic acids and more generally to any purification or detection method that relies on the hybridization of complementary nucleic acids for selectivity. For example, the improved hybridization methods herein can be used to bind to and extract target nucleic acids from a mixture, for diagnostic assays that rely of the identification and analysis of one or more nucleic acids and for various genetic assays for the detection of genetic targets such as genes, gene fragments, bacteria, viruses and other microorganisms. The present invention introduces methods providing for enhancing the hybridization selectivity of devices that use immobilised nucleic acids on a substrate for selective detection of target nucleic acids by controlling the density and organisation of immobilization of oligomers including nucleic acids on the substrate.

The invention includes any substrates for use in any purification method which relies on nucleic acid hybridization for selectivity or any hybridization assay, comprising a plurality of first nucleic acids immobilized on the substrate alone or in combination with other oligomers in a medium-high or high immobilization density. One skilled in the art can determine a suitable number of contiguous matching nucleotides necessary for obtaining hybridisation for example, there may be 5, 6, 7, 8, 9, 10, 15, 18, 20, 25 or more contiguous, matching nucleotides. The methods of this invention and the substrates having medium-high or high immobilization density herein can provide for enhanced selectivity and sensitivity in assays of relatively short nucleic acids, e.g., those having 20 bases or less and more particularly those with about 10-12 bases. The methods and substrates of this invention are particularly useful for SNP (single nucleotide polymorphism) analysis.

In a specific embodiment, the invention relates to substrates for conducting hybridizations which comprise a first immobilized layer of oligomers at medium-high to high immobilization density and which comprises at least one first immobilized nucleic acid over a portion of its surface and a second immobilization layer of oligomers at low immobilization density and which comprises the at least one first immobilized nucleic acid over a portion of its surface. The immobilized layers of different density are spatially discrete and do not overlap on the substrate. Hybridization to the medium-high to high density immobilization layer will be more selective than hybridization to the low immobilization layer density layer. These substrates can be employed in hybridization assays, particularly when high ionic strength samples or high ionic strength washes are employed, when a low selectivity hybridization control is desired. This type of substrate can be used to detect and distinguish a selected target nucleic acid in a sample and nucleic acids in the sample that exhibit small differences (1 or several) in sequence from the selected target nucleic acid. The invention also provides a combination of two substrates one having a medium-high to high immobilization density of oligomers including a first nucleic acid and a second substrate having a low immobilization density of oligomers including the first nucleic acid.

In another specific embodiment, the invention relates to substrates for conducting hybridizations which comprise a plurality of spatially discrete immobilized layers of oligomers at medium-high to high immobilization density on the substrate surface wherein at least a portion of the spatially discrete immobilization layers comprise different immobilized nucleic acids. In a particular embodiment, nucleic acids that each differ from one another by a single base change are immobilized in different spatially discrete immobilization layers on the substrate. In another particular embodiment, nucleic acids that each differ from one another by a single base change at a selected position in the nucleic acid sequence are immobilized in different spatially discrete immobilization layers on the substrate. These substrates can be employed in hybridization assays, particularly when high ionic strength samples or high ionic strength washes are employed, when it is desired to identify or quantitatively assay sample nucleic acids that differ by a single base. The invention also provides a plurality of substrates having a medium-high to high immobilization density of oligomers wherein each different substrate comprises a different immobilized nucleic acid and wherein the different immobilized nucleic acids differ from one another by one base or that differ from each other by one base at a selected position in the sequence of the nucleic acid.

Other specific embodiments of the invention relate to a substrate for hybridization, comprising a plurality of first nucleic acids immobilized on the substrate and a plurality of oligomers other than nucleic acids immobilized on the substrate. It will be apparent to a skilled artisan how to adapt the teachings in this application for use with oligomers other than nucleic acids. The oligomers other than nucleic acids can be similar or different in length from the nucleic acids with which they are co-immobilized. Oligomers other than nucleic acids that can be used in preparation of these substrates can be linear or branched in strucutre and can include with out limitations polyethers which may be linear or branched. The relative amounts of nucleic acids to oligomers that are not nucleic acids that are immobilized on a substrate can vary widely. The number ratio (or molar ratio) of nucleic acids to other oligomers can, for example, vary from 1:1 up to 1:1000. In specific embodiments this ratio can be 1:10, 1:20, 1:50, 1:100 or 1:500.

Another aspect of the invention is a method for preparing a substrate for a hybridization assay, comprising the steps of immobilizing a plurality of first nucleic acids alone or in combination with oligomers that are not nucleic acids on the substrate at a medium-high or high immobilization density.

Another embodiment of the invention relates to a method of hybridizing nucleic acids comprising the steps of:

providing a substrate comprising a plurality of first nucleic acids immobilized on the substrate, the plurality of first nucleic acids having a medium-high or high immobilization density on the substrate; and

contacting the substrate with at least one second nucleic acid having a region of contiguous nucleotides that are complementary to all or part of at least one of the first nucleic acids, so that the second nucleic acid hybridizes to the at least one first nucleic acid.

Any of the substrates of this invention comprising an immobilization layer having a medium-high or high immobilization density of oligomers including nucleic acids over at least a portion of its surface can be used in similar methods of hybridizing nucleic acids. Additionally, the invention relates to the use one or more substrates of the invention in a hybridization assay for diagnosing a disease state or detecting a genetic target (e.g., a virus, a bacterium or a gene). The invention also includes a kit for detecting the presence of a genetic target in a test sample, comprising a substrate of this invention, optionally in association with a hybridization buffer.

The invention also provides a method for detecting the presence of one or more genetic targets in a test sample, comprising the steps of:

providing a substrate comprising a plurality of at least a first genetic marker nucleic acid immobilized on the substrate alone or in combination with one or more oligomers that are not nucleic acids, such that the immobilization density of oligomers on the substrate is medium-high or high immobilization density;

contacting the substrate with a test sample comprising a mixture of nucleic acids so that a second nucleic acid that may be in the test sample which has a region of contiguous nucleotides that are complementary to all or part of the at least one first genetic marker nucleic acid hybridizes to the genetic marker nucleic acid; and

detecting hybridization of the immobilized genetic marker nucleic acid to the second nucleic acid, wherein the detection of hybridization is indicative of the presence of a genetic target in the sample.

In a preferred embodiment of the hybridization and assay methods herein employing a substrate a medium-high or high immobilization density of oligomers including a plurality of first nucleic acids, a second nucleic acid having a region of contiguous nucleotides that is complementary to all or part of at least one of the first nucleic acids will selectively hybridize to the at least one first nucleic acid. Also, preferably, in an in-vitro assay, the difference in Tₘ between

(i) a fully-matched complex immobilized to the substrate, the complex comprising the first nucleic acid and a second nucleic acid which has a sequence complementary to the first nucleic acid; and

(ii) a mismatch complex immobilized to the substrate, the complex comprising the first nucleic acid and a second nucleic acid having a single nucleotide mismatch;

is not decreased, and preferentially increased, over the difference in Tₘ between the complexes immobilized at low immobilization density. Preferably, the mismatched nucleotide on each nucleic acid is proximate to the middle of the hybridized portion of each nucleic acid. Preferably, the difference in Tₘ is at least 5 degrees Celsius.

In the methods of this invention, a second nucleic acid and the at least one first nucleic acid are optionally hybridized in a high ionic strength solution.

In a preferred embodiment, the hybridisation comprises a Tₘ inversion effect. In a further preferred embodiment, the interfacial hybridisation for matched systems of nucleic acids exhibits enhanced sensitivity to temperature.

The hybridization substrates and hybridization methods and assays of this invention have application to myriad device platforms and fields of application including human and veterinary in-vitro diagnostics, environmental monitoring, food microbiology, food varietal identification, and biowarfare screening applications.

For example, the Defence Advanced Research Projects Agency (DARPA) has invested millions of dollars in biotechnology companies over the past several years, hoping to identify biowarfare agents to facilitate prevention, neutralization, or to reverse the effects of such agents. Identifying these agents (e.g. small pox virus, anthrax bacteria or spores, level 3 or 4 biohazard agents) is an important part of successfully implementing timely countermeasures. Testing for genetic identity in crops has also entered the limelight of biotechnology endeavours wherein much debate over the presence and safety of genetically modified organisms (GMOs) in food and feed commodities is ongoing and has fuelled the need for reliable testing for GMOs. Identification of food pathogens and contaminants in the forms of viral, bacterial or fungal agents (e.g. salmonella, lysteria, and E. coli) will be valuable for prevention and treatment programs for food safety assurance and will be required at all levels of food supply chains. Environmental testing applications include water quality testing (e.g. E. coli 0157, cryptosporidium, giardella), soil contamination, and tracking of populations of indicator or biorecovery organisms. Genomics applications can include the design and monitoring tools for new biotechnology products and breeding programs, as well as detection of up-regulated and down-regulated genes.

Applications of the present invention include, but are not limited to, the detection of pathogenic bacteria, viruses, fungi or polymorphisms in genetic sequences. The assay would preferably begin by preparation of a substrate via medium-high to high-density immobilisation of an immobilization layer containing one or more probe nucleic acid suitable for given application to detect one or more selected target nucleic acids. The immobilization layer can contain immobilized probe nucleic acid alone or in combination with other immobilized oligomers that are not nucleic acids. The medium-high to high-density immobilisation substrate would then be exposed to a solution of nucleic acids derived from a sample (which may contain target nucleic acids) under conditions that support hybrid formation between nucleic acids of the sample that are complementary to the probe nucleic acids immobilised on the substrate and thereafter hybridization is detected. The enhancement in selectivity offered by the method and substrates of the present invention serves to improve the confidence in positive detection and reduction of false results, owing to more selective binding to the probe nucleic acid. This improved selectivity is manifested in enhanced signal-to-noise ratios observed in true positive results. Enhanced signal-to-noise facilitates signal processing, especially in applications based on the use of microarray platforms, to reduce the complexity of data treatment and to reduce the redundancy that is typically required to effect mismatch discrimination.

In diagnostics, the tested sample does not have to use the same nucleic acids that were taken from a patient. The sample can be amplified prior to hybridization analysis by PCR or other such commonly employed methods. In addition, PCR or other such amplification methods can also be used to generate copies of the nucleic acid sequences in samples that incorporate detectable labels (e.g. fluorophores or other moieties that can serve to permit attachment of a label or a signalling chemistry). This type of labelling is commonly done for sample preparation prior to analysis using a nucleic acid microarray. The nucleic acid in any test samples from any environment (e.g., food samples, plant materials, water samples etc.) can be amplified prior to hybridization analysis. Furthermore, it will be appreciated by those of ordinary skill in the art that test samples may be subjected to various purification steps that are compatible with retention of potential target nucleic acids prior to hybridization analysis.

Some methods of analysis can use an indicator other than an indicator agent. For example, surface plasmon resonance sensors function by monitoring changes in optical mass at the interface brought about by binding of the target; acoustic wave devices monitor changes in viscoelastic coupling between the substrate and the ambient owing to binding of the target at the interface. There are also electrochemical methods of nucleic acid analysis that do not require the use of an indicator agent.

The methods of this invention control the selectivity of binding of nucleic acid on the surface of nucleic acid diagnostic or microarray entities that make use of immobilized nucleic acids as the chemically selective recognition element, by means of control of the immobilization density and organisation of nucleic acids alone or in combination with oligomers that are not nucleic acids on the substrate surface.

Dramatically improved hybridization selectivity has been observed in assays performed at high ionic strength and in which probe nucleic acids, alone or in combination with oligomers that are not nucleic acids, are immobilized at medium-high or high immobilization density, i.e. an inversion effect at high ionic strength. Normally, the difference in Tₘ between fully complementary and partially complementary hybrids decreases as the ionic strength in the solution in which hybridization occurs is increased, since the increasing salt concentration shields the phosphate anion backbones of the hybridized strands from each other, reducing electrostatic repulsion. In experiments detailed herein, an unexpected inversion effect was observed in which the difference in Tₘ between fully complementary and partially complementary hybrids did not decrease as the ionic strength of sample solutions increases when hybridization is conducted with probe nucleic acids immobilized at medium-high to high immobilization densities on substrates. This inversion effect is also observed when a mixture of probe nucleic acids and oligomers that are not nucleic acids are immobilized at medium-high to high immobilization densities on a substrate. This inversion effect results in the maintenance of selectivity or in improved selectivity in hybridization assays performed in the presence of high ionic strength when the assays employ substrates carrying probe nucleic acids in a medium-high or high density immobilization layer.

Control of oligonucleotide immobilization density and organisation in devices that make use of covalently immobilized nucleic acids may be achieved by control of the number of available reactive sites on a substrate onto which the oligonucleotides and any oligomers that are not nucleic acids will be immobilized. Since it is desirable to immobilize the nucleic acids to the substrate surface via appropriate linker molecules (e.g., polyether or hydrocarbon chains [11, 24]), control of immobilization density can be afforded through control of the immobilization density of linker molecules, however other methods can be employed to control immobilization density. An exemplary method for controlling immobilization density control is by control of the density of polyether linker moieties on a substrate Preferred polyether type linker molecules are greater than about 20 and less than about 40 atoms in length [M. S. Shchepinov, S. C. Case-Green, and E. M. Southern, Nucleic Acids Research, v. 25, 1997, p. 1155]. Linker structures can also include dendritic forms of poly(ethylene oxide) chains, such as have found application in the preparation of nucleic acid microarray substrates [M. Beier and J. D. Hoheisel, Nucleic Acids Research, v. 27, 1999, pp. 1970-1977]. Linkers can also be hydrocarbon based and more preferable contain electronegative moieties within them, such as oxygen, to minimize associative interactions [S. L. Beaucage and R. P. Iyer, Tetrahedron, v. 48, 1992, pp. 2223-2311]. Linkers N are preferably longer than 18 atoms [S. L. Beaucage and R. P. Iyer, Tetrahedron, v. 48, 1992, pp. 2223-2311]. References in this application to the nucleic acid:linker refer to a situation where a nucleic acid is tethered to a substrate by a linker as well as the situation where there is no linker and the nucleic acid is immobilized directly on the substrate.

Substrates useful in the methods of this invention include any solid material that can be employed to immobilize nucleic acids, either directly or through a linker and that is compatible with hybridization of nucleic acids. Substrates can be made, for example, of glass, quartz, metals (including gold and silver) and organic or inorganic polymers (e.g., plastics) and can have a variety of shapes, e.g., plates, tubes, beads, etc. Substrates also include optical elements such as waveguides and optical fibres such as those employed in optical biosensors.

The selectivity and sensitivity of hybridization assays exemplified herein were performed using nucleic acid biosensors with controlled immobilization densities of oligonucleotides alone or in combination with oligomers that are not nucleic acids. The measurement technique employed was based on total internal reflection fluorescence (TIRF), which has been described in detail [28]. Thermodynamic selectivity and the thermodynamic stability of hybrids formed in an interfacial environment were examined by use of thermal denaturation profiles collected using this instrument. These profiles provided the necessary information to determine thermodynamic parameters such as the thermal denaturation temperature (Tₘ, or temperature at which 50% of all duplexes formed are denatured), van't Hoff enthalpy change (ΔH_{VH}) and standard enthalpy change (ΔH°) of the denaturation transition.

Selectivity of hybridization is usually affected by control of solution conditions such as temperature and ionic strength in such a way as to minimize the energetic stability of hybrids with partially complementary sequences, relative to that of the fully complementary hybrid. This is achieved by manipulating solution conditions such that differences in Tₘ between fully complementary hybrids and partially complementary hybrids are maximized. This then facilitates maximum selectivity by doing the hybridization assay at temperatures above the Tₘ of the partially complementary hybrids and below that of the fully complementary hybrids.

The effect of ionic strength and immobilization density and organisation on the Tₘ values of immobilized oligonucleotide hybrids was examined. It was found that immobilized oligonucleotide hybrids possess reduced thermodynamic stability relative to analogous systems observed in bulk solution. It was also found that increasing the oligonucleotide immobilization density to the point where interactions between neighbouring nucleic acids and/or other oligomers became probable resulted in a reduction of the thermodynamic stability of interfacial nucleic acid hybrids formed. The reduction in stability of nucleic acid hybrids as a result of immobilization served to amplify differences in thermodynamic stability between fully complementary hybrids and partially complementary hybrids. Consequently, control of thermodynamic selectivity of hybridization occurring in an interfacial environment is tuned by controlling the density and organisation of immobilized nucleic acids and other oligomers such that interactions between nearest neighbours are controlled. These interactions are not purely a function of nucleic acid number density, but are also related to the extent to which nearest neighbours can interact. As a result, tuning the selectivity considers the length, molecular structure and conformation, any extended solvent structure (e.g., hydration structure) and the effect of solvation and or electrostatic fields of the immobilized nucleic acid and other oligomers and their mean separation distance. The effect of these parameters on the ability of nearest neighbour oligomers to interact is estimated by considering the ratio of the average separation of immobilized oligomers to the average length of the immobilized oligomers as is discussed above.

The invention finds direct application with biosensor and microarray technologies that make use of immobilized nucleic acids including nucleic acid analogues for purposes of simple screening, sequence determination, or quantitative transduction of nucleic acid or nucleic acid analogue binding. The use of automated oligonucleotide synthesis facilitates the immobilization of nucleic acids, nucleic acid analogues or other oligomers in high density, which imparts greater selectivity of binding of nucleic acids or nucleic acid analogues.

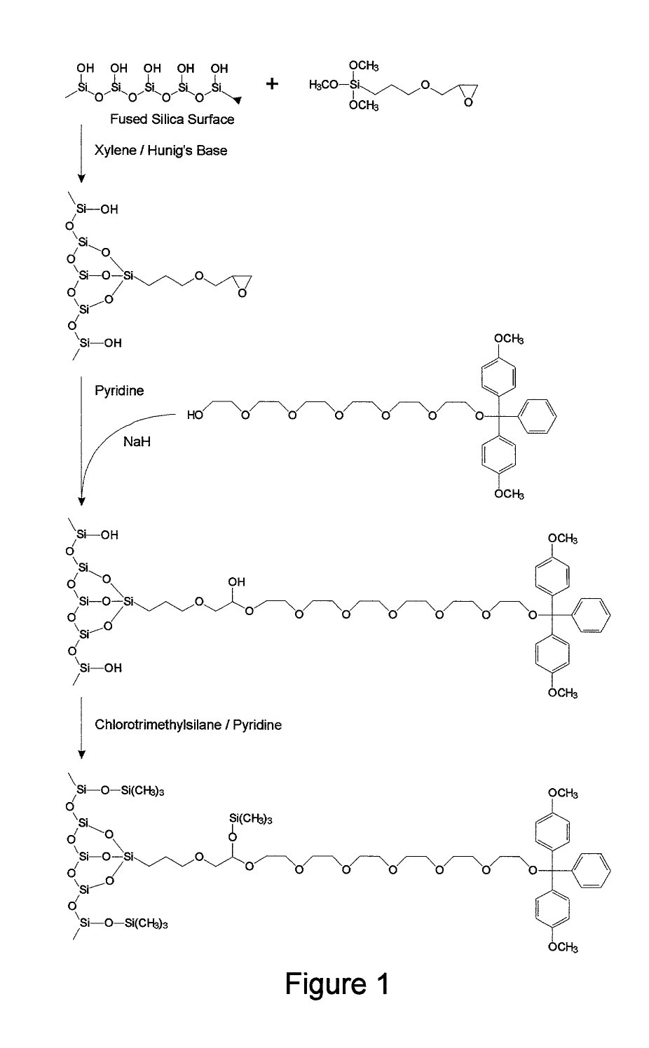

The invention is useful with any substrates to which nucleic acids can be bound directly or indirectly via a linker(for example, glass or fused silica surfaces). The reaction scheme shown in FIG. 1 and the examples show types of nucleic acid binding to substrate via linker molecules. Other strategies for achieving such immobilisation are known. For example, with plastic substrates, the surface of the plastic could be hydroxylated via gas plasma reaction chemistry in an oxygen rich environment and then the same chemistry as that used for silica-based surfaces can be used for immobilization. Alternatively, the hydroxyl terminus of the linker molecule could be triflate-activated and then exposed to the hydroxylated plastic surface for controlled reaction periods to permit controlled coupling and surface density of linker molecules that in turn template the sites for oligonucleotide attachment and the surface packing density of immobilised nucleic acid.

In another example, the plastic surface can be aminated by gas plasma reaction chemistry in a nitrogen rich environment. Phosphoramidite synthons of the linker molecules could then be immobilised in controlled fashion through control of reaction conditions (e.g., reaction time, reactant concentration, temperature, and/or choice of solvent conditions) which in turn would provide template sites for oligonucleotide attachment. This can be done, for example, on either hydroxylated or aminated plastic substrates. The general chemistry for attachment of phosphoramidite synthons of the linker molecule to a hydroxylated or aminated surface would preferably follow the well-established solid-phase β-cyanoethylphosphoramidte chemistry as used for nucleic acid synthesis in DNA synthesizers.

On gold substrates, sulphur terminated oligonucleotide-linker conjugates can be employed that bind to the gold surface via the well-established gold-sulphur coordinate interaction.

The density of immobilised oligonucleotide will largely be governed by self-assembly processes and so introduction of a mixture of sulphur terminated oligonucleotide-linker conjugate and a mercapto-terminated short length co-reactant molecule can be applied to the surface wherein the ratio of the oligo-linker conjugate to co-reactant will control the mean separation distance between neighbouring immobilised oligonucleotides or oligomers. Furthermore, the chemistry at the terminus of the co-reactant oriented away from the surface can be selected to control the physical chemistry of the surface (surface free energy) such that the extent and energetics of interactions between the immobilised oligonucleotide, oligomers and the exposed surface can be controlled. This will also have ramifications on interactions between the surface and any species in solution, thereby facilitating control of non-selective adsorption of oligonucleotides or any other species which may give rise to false positive signal generation or other undesired alterations in interfacial free energy.

In specific embodiments, the substrate is as described in this application, with the proviso that the substrate does not include first nucleic acids immobilized on gold. In other specific embodiments, the substrate is as described in this application with the proviso that the substrate is not an optical element, such as an optical waveguide or an optical fibre.

In order to characterize the effects of oligonucleotide immobilization density and organisation on the thermodynamics of hybridization, the following classifications of immobilization density have been defined hereinabove: low, medium, medium-high and high. It can be predicted that increasing the immobilization density from low to medium to high immobilization density will likely result in a more homogeneous distribution of oligomer orientations, with maximal oligomer extension away from the substrate surface being achieved with higher immobilization densities.

Medium-high to high density films containing nucleic acids immobilized on substrates greatly enhance the ability to detect preferential hybridisation of a single base pair mismatch. Configurations employing these medium-high to high density immobilized films in diagnostic instrumentation include:

A cartridge system of biosensors or substrates(each cartridge containing a single fibre or a single substrate) for use in single nucleotide polymorphism (SNP) detection having a four cartridge system containing

1. a cartridge containing a fibre or substrate having a common sequence immobilized to normalize for total amount of DNA in the sample;

2. a cartridge containing a fibre or substrate having a wild type sequence immobilized to monitor the non-mutated version of the gene-fragment under investigation;

3. a cartridge containing a fibre or substrate having a SNP sequence immobilized containing the mutated version of the gene-fragment;

where in the fibres or substrates of 1-3 the immobilization layer is at medium-high to high density; and

4. a cartridge containing a non-specific fibre or substrate to control for adsorption phenomenon and photobleaching of the dye used for detection of hybridization.

Additional SNP detection chambers can be added in pairs of cartridges: one for the wild type sequence for the gene-segment and another for the gene-segment including the SNP.

A number of clinical tests require the identification of a specific organism from within a background of a group of organisms which have a similar genome composition. This is especially relevant to virology. In these cases identification of the correct organism depends on designing a probe in a region of the genome where there is a significant degree of difference between the organisms. Viruses quite often contain DNA or RNA sections that are hypervariable, and the genome of a virus is comparatively small compared to other organisms. This can make selection of suitable target sequences difficult, since target choices are limited to regions where there are small stretches of variances between different viral strains. Distinguishing such small differences necessitates the use of an instrument which is rapidly able to quantitatively distinguish between organisms exhibiting small stretches of differences in their genome. The configuration of this instrument would be similar to that described above for SNP analysis, except that additional chambers are likely not required for this purpose.

A number of diagnostic tests utilize amplification of genes using PCR followed by digestion with a restriction enzyme wherein PCR amplification is used to introduce mutated bases pairs to form the target sequence of the restriction enzyme. The diagnostic test requires that a restriction enzyme site be formed or destroyed as a result of the mutation. This does not always happen and therefore it becomes difficult to design a diagnostic test. The methods and substrates of this invention permit the direct detection of sequences with as few as a single mutation. Once again, a cartridge format of fibres or substrates as outlined above could be used.

The differences observed in hybridization efficiencies between single based pair mismatched (SPBM) sequences immobilized in low and high density films indicate that at low densities the hybridization signal will become less discriminatory as the salt concentration is increased. In contrast the hybridization signal with high density films will retain and preferably become more discriminatory with increasing salt concentration. This implies that for a SBPM immobilized at low density a detectable hybridization signal (e.g. fluorescence signal indicative of hybridization) would increase if the concentration of salt were increased over time. This would result from increased non-specific binding in the low density layer. For the same sequence immobilized at high density the detectable hybridization signal would exhibit a slight decrease in the fluorescence signal with increasing salt concentration using sequences which had a SBPM. If the hybridization signals for the low and high density substrates having the same attached sequence (with a SBPM to a sample), are subtracted the difference in fluorescence signal will be significantly greater than if there was an exact sequence match between the attached and target sequences. This difference in signal can serve as a basis for the detection of nucleic acids having single base pair mismatches. In this application, a cartridge format as described above can be used except that two of the cartridges would employ a fibre or substrate with a low density immobilization layer and the other two cartridges would employ a fibre or substrate having a high density immobilization. One cartridge at each density would contain the potential SBPM sequence, and the other cartridge at each density would contain the wild type sequence. By performing a ratiometric analysis from the signals originating from the two SBPM or wild type chambers, the presence of the SBPM sequence in a sample can be detected and the quantity of the SBPM sequence in samples could be monitored.

### EXAMPLES

The present invention is further illustrated by the following specific examples, which are not intended in any way to limit the scope of the invention.

### Example 1

### Control of Oligonucleotide Immobilization Density by the GOPS-HEG Method: Low Density Case

1.1: Chemicals.

Unless otherwise noted, all reagents for syntheses were obtained from commercial suppliers (Aldrich, Milwaukee, Wis., USA or Lancaster Synthesis Inc. Windham, N.H., USA) and were used without further purification. Unless otherwise noted, all solvents were EM Science brand (distributed by VWR Canlab, Mississauga, ON, Canada) and of reagent grade. Solvents were further purified and/or dried, when necessary, by standard distillation methods. Acetonitrile was biosynthesis grade low water from EM Science (VWR Canlab). Tetrahydrofuran (THF) was distilled from sodium/benzophenone ketyl under argon. Dichloromethane was pre-dried by stirring with calcium chloride overnight followed by distillation over calcium chloride under argon. Acetone was distilled over calcium sulphate under argon. Nitromethane was dried over calcium chloride. Molecular biology grade salts were purchased from EM Science. Molecular biology grade polyacrylamide gel electrophoresis reagents and apparatus were obtained through Bio-Rad (Hercules, Calif.). DNA synthesis reagents were purchased from Dalton Chemical Laboratories Inc. Sterile water for use on its own and with hybridization buffer was produced from a Millipore Gradient A10/Elix5 purification system, then subsequently treated with diethyl pyrocarbonate (Aldrich) and sterilized by autoclave. Control pore glass was obtained from CPG Inc. (Lincoln Park, N.J., USA) and had a mean pore diameter of 515 Å, specific surface area 43.5 m²/g, and a particle size of 125-177 microns. All glassware was pre-dried prior to use and reactions involving moisture-sensitive reagents were executed under an inert atmosphere of dry argon or nitrogen. Flash chromatography was performed using silica gel 60 (Toronto Research Chemicals, 230-400 mesh ASTM).

1.2: Instrumentation

All reactions requiring an inert and anhydrous atmosphere were done in a NEXUS glove box equipped with a solid-state water probe (Vacuum Atmospheres, CA). The water content of the nitrogen atmosphere within the glove box was maintained at <1ppm at all times. An Agilent 1100 HPLC with ChemStation control software, quaternary pump, online degasser, auto sampler, thermostated column compartment and diode array detector was used for sensor quality control analysis of oligonucleotide and polyelectrolyte products. Water determinations were done by use of an AquaStar™ C-400 titrator (EM Science). Oligonucleotide and polyelectrolyte syntheses were done using an ABI 394 DNA/RNA synthesizer (PE Biosystems, Foster City, Calif.). An Agilent 8453 UV-vis spectrophotometer with ChemStation control software was used for all UV-VIS absorbance measurements. ¹H-NMR spectra were recorded on a Varian 200Gemini NMR. For ¹H-NMR spectra run in CDCl₃, chemical shifts (δ) are reported in parts per million relative to the internal standard tetramethylsilane (TMS). All NMR couplings are given in Hz. Abbreviations s, d, t, q, qt, m and br are used for singlet, doublet, triplet, quadruplet, quintuplet, multiplet and broad, respectively. Electron impact spectra (EI) were obtained on a Micromass 70-S-250 mass spectrometer.

1.3: Preparation of Fused Silica Optical Fibre Pieces

The jacket material surrounding the fused silica optical fibres (400 μm core diameter, 3M PowerCore™ Series Optical Fibre, FT-400-URT or FP-400-UHT, distributed by Thor labs) was mechanically removed by use of a fibre stripping tool (Thor Labs Inc.) to reveal the fused silica core material and cladding layer. Optical fibre pieces 48 mm in length were then made by use of a custom built diamond edged fibre scoring device. The fibre scoring device consisted of a chisel-edged diamond pencil secured in a spring loaded rail assembly situated on a rotating platform. The rotating platform surrounded a centrally mounted pin-chuck that was used to hold the base of the optical fibre segment to be scored. An adjustable Teflon® stop, juxtaposed to the diamond pencil and in contact with the optical fibre was used to prevent the pressure applied by the spring-loaded diamond pencil from snapping the brittle unjacketed portion of the optical fibre. The diamond pencil/rail assembly was rotated about the optical fibre to provide a uniform score about the circumference of the optical fibre. An optical fibre segment with a cleanly cleaved terminus of good optical quality was then created by pulling the top portion of the scored fibre away from the remainder of the optical fibre secured in the pin-chuck. The termini of the fibre pieces were visually inspected at 40× magnification beneath an optical microscope to ensure the fibre termini were flat, orthogonal to the length of the fibre, and free of chips and nicks.

1.4 Cleaning of Substrates Prior to Surface Modification

The glass or fused silica substrates were immersed in a 1:1:5 (v/v) solution of 30% ammonium hydroxide/30% hydrogen peroxide/water and the mixture was gently agitated at 80° C. for five minutes. The substrates were then removed, washed with copious amounts of water and then treated with 1:1:5 (v/v) conc. HCl/30% hydrogen peroxide/water for five minutes at 80° C. with gentle agitation. The substrates were then sequentially washed with water, methanol, chloroform and diethyl ether, respectively, and dried in vacuo at 130° C. for 16 hours followed by storage under an anhydrous atmosphere (<1 ppm water) until required.

1.5: Functionalisation of Solid Substrates with 3-Glycidoxypropyltrimethoxysilane (GOPS).

The cleaned solid substrates (fibres and CPG) were suspended in a solution of xylene/3-glycidoxypropyltrimethoxysilane (GOPS)/diisopropylethylamine (500:28:1 v/v/v, total water content 22.8 ppm). The reaction was stirred under an anhydrous atmosphere at 80° C. for 24 hours. The substrates were then collected and twice washed with two 200 ml portions of methanol, dichloromethane and diethyl ether, respectively, and then dried and stored in-vacuo at room temperature until required.

1.6: Synthesis of 17-Dimethoxytrityloxa-3,6,9,12,15-pentaoxa-1-heptadecanol (DMT-HEG).

A solution of dimethoxytrityl chloride (7.1 g, 21 mmol) in dry pyridine (10 ml) was added in a drop-wise fashion to a stirred solution of hexaethylene glycol (5.6 ml, 21 mmol in 5 ml pyridine) under an argon atmosphere and over a duration of ca. 1 hour. Stirring was continued overnight after which time the reaction mixture was combined with dichloromethane (50 ml). The mixture was shaken against 5% aqueous bicarbonate (2×900 ml) and then with water (2×900 ml) to remove unreacted HEG, pyridine and salts. The organic layer was recovered and dried under reduced pressure to yield the crude product as a pale yellow oil. The product was purified by liquid chromatography on a silica gel column eluted with 1:1 dichloromethane/diethyl ether containing 0.1% triethylamine (2.9 g, 24% yield). ¹H NMR (200 MHz, CDCl3)d: 7.47-7.19 (m, 9H), 6.81 (d, 4H, J=8.8 Hz), 3.78 (s, 6H), 3.74-3.51 (m, 22H), 3.22 (t, 2H, J=5.8 Hz), purity (HEG-DMT)=96%.

1.7: Linkage of DMT-HEG to GOPS Functionalised Silica Substrates.

DMT-HEG (10 eq. relative to the quantity of surface hydroxyl moieties, 700 mg DMT-HEG/100 mg CPG) that had been dried by extended storage in-vacuo (>72 hrs.) was dissolved in 20 ml of anhydrous pyridine and introduced to an excess of NaH (10 eq.) that had been thrice washed with dry hexane to remove the oil in which it was suspended. The reaction was permitted to proceed with stirring for 1 hour at room temperature under an argon atmosphere. The reaction mixture was filtered through a sintered glass frit under a positive pressure of argon and the filtrate immediately introduced to the reaction vessel containing the GOPS functionalised substrates. For the case where optical fibre substrates were to be functionalised with the HEG-based linker molecules, an addition 10 ml of anhydrous pyridine was introduced to the reaction vessel so that the substrates were completely immersed. The DMT-HEG coupling reaction was permitted to proceed under a positive pressure of argon gas at room temperature with gentle agitation on an oscillating platform stirrer for a duration of 1 hour. Following the coupling reaction, the substrates were recovered by filtration over a fritted glass funnel and washed with 150 ml portions of methanol, water, methanol, and diethyl ether, respectively, to remove non-specifically adsorbed reactants. The DMT-protected HEG functionalised substrates stored in-vacuo until required.

1.8: Solid Phase Phosphoramidite Synthesis of Oligonucleotides

Oligonucleotide synthesis was done using the manufacturer-supplied synthesis cycles modified to increase the delivery times of the reagents as required to completely fill the synthesis columns that were used. Oligonucleotide synthesis onto optical fibres (400 mm i.d.×48 mm) was done in a custom manufactured Teflon synthesis column (6 mm i.d.×50 mm) capable of holding 8 fibres in an evenly distributed and non-contacting fashion via cylindrical bores (400 mm i.d.×2 mm deep) machined into one of the end caps. Synthesis on CPG was done in Teflon® columns that were designed as mimics of the 0.2 μmol columns (8 mm i.d×10 mm) supplied by ABI using Teflon® end filters (0.22 μm pore size, PE-ABI) to contain the glass beads within the column. All column end-caps were secured onto the column bodies by use of aluminium crimp seals. Synthesis of oligonucleotides on nucleoside functionalised LCAA-CPG substrates was done in polyethylene columns as supplied by the manufacturer. Detritylation was done using with 2% dichloroacetic acid in dichloroethane.

1.9: Cleavage Oligonucleotides from CPG Supports

Cleavage of oligonucleotides from CPG supports was achieved by standing the oligonucleotide functionalised substrates in 30% aqueous ammonia at 55° C. for 16 hours. In the case where quantitative determinations of oligonucleotide assembly were required, a known quantity of standardised carrier oligonucleotide was applied to the substrates prior to ammonia treatment so that sample loss could be corrected for. Following incubation, the ammonia solution containing the liberated oligonucleotides was flash-frozen in liquid N₂ and the solvent was removed under reduced pressure in a centrifugal evaporator. The residue containing the deprotected oligonucleotides was then stored dry at −20° C. until required.

1.10: Anion-Exchange HPLC (AE-HPLC) Investigations of Cleaved Oligonucleotide-Linker Conjugates

AE-HPLC analysis of oligonucleotides was done using a Perkin-Elmer Series 400 solvent delivery system coupled to a Rhyeodyne model 7125 injector (Rhyeodyne Inc., Cotati, Calif., USA) fitted with a 6 μl injection loop. The chromatographic column used for investigations of oligonucleotides assembled on CPG and fused silica substrates was a Waters Gen-Pak FAX column (4.6 mm i.d.×100 mm, Waters, Milford, Mass., U.S.A.) that contained a polymer-based packing material composed of nonporous particles of 2.5μm diameter functionalised with diethylaminoethyl (DEAE) functional groups. The column temperature was maintained at 30° C. by use of a water jacket (Alltech, Deerfield, Ill., U.S.A.) in combination with a thermostated bath )mgw M3, Lauda-Königshofen, FRG). Detection was done spectrophotometrically by monitoring eluent absorbance at 260 nm using a single-wavelength Perkin-Elmer LC-95 TV/VIS detector (Perkin-Elmer, Norwalk, Conn., U.S.A.). Data were acquired and processed with a HP integrator (Hewlett Packard). The mobile phase was delivered to the column at a flow rate of 0.5 ml-min⁻¹. A gradient elution protocol modified from that supplied by the manufacturer was employed and is detailed in Table 1. The two main solvent systems used for oligonucleotide separations were: Buffer A—25 mM TRIS and 1 mM EDTA in 10% aqueous acetonitrile (pH=8.0, adjusted using 0.5 M sodium hydroxide solution) and Buffer B—same composition as Buffer A with sodium chloride added to a concentration of 1.0 M. All solvents were degassed by vacuum-filtration through a 0.2 μm nylon membrane filter prior to use.

TABLE 1
AE-HPLC Elution Profile for Separation of Oligonucleotides

| | | ELUTION | DURATION | |
|---|---|---|---|---|
| STEP | MOBILE PHASE COMPOSITION | METHOD | (MIN.) | PURPOSE |
| 1 | 90% Buffer A, 10% Buffer B | Isocratic | 5 | Sample |
| | 90% Buffer A, 10% Buffer B | | | Introduction |
| 2 | to | Linear Gradient | 30 | Separation |
| | 40% Buffer A, 60% Buffer B | | | |
| 3 | 100% Buffer B | Isocratic | 5 | Washing |
| 4 | 33 mM Phosphoric Acid | Isocratic | 5 | Washing |
| 5 | 100% Buffer B | Isocratic | 5 | Washing |
| 6 | 33 mM Phosphoric Acid | Isocratic | 5 | Washing |
| 7 | 100% Buffer B | Isocratic | 5 | Washing |
| 8 | 90% Buffer A, 10% Buffer B | Isocratic | 30 | Conditioning |

1.11: Discussion

This example of a method to control the delivery of linker molecules to an activated surface for coupling is a diffusion dependent phenomenon. Consequently, all reactions were done with an excess of linker molecules, with linker immobilization yield or density control then facilitated through control of the conditions and duration of the coupling reaction. The methods used for descriptive purposes of this invention made use of hexaethylene glycol (HEG), protected at one terminus with dimethoxytrityl (DMT) to yield a monofunctional linker molecule, but the physical properties of immobilized nucleic acids which impart the selectivity observed includes, but is not limited to use of, this linker system in the immobilization process. The immobilization of polythymidylic acid icosanucleotides (dT₂₀) onto the surface of fused silica optical fibres and controlled pore glass (CPG) substrates was achieved by means of a modification to the method of Maskos and Southern [24]. The substrates were first functionalized with glycidoxypropyltrimethoxysilane (GOPS). Hexaethylene glycol (HEG), protected on one terminus with dimethoxytrityl (DMT) groups in order to ensure single-site reactivity and to minimize the risk of formation of closed-ring structures, was then covalently attached to the epoxysilane layer. This reaction scheme is seen in FIG. 1. A batch of the GOPS-functionalized substrates underwent the DMT-HEG coupling reaction for a duration of 1 hour.

The nucleic acid biosensors described herein made use of automated β-cyanoethylphosphoramidite chemistry to synthesize the immobilized oligonucleotides directly onto DMT-HEG functionalized substrates. While the nucleic acid biosensors described used fused silica optical fibres as the substrates onto which the HEG-oligomer conjugates were immobilized, HEG-oligomer conjugates were also synthesized on controlled pore glass (CPG), which has a large, well-defined surface area. This was done in order to provide a significant yield of the immobilized species which could be recovered and analyzed independently by anion-exchange high performance liquid chromatography (AEHPLC), to provide information with respect to the yield and quality of HEG-oligomer synthesis, and serve as a screening method for unwanted side products.

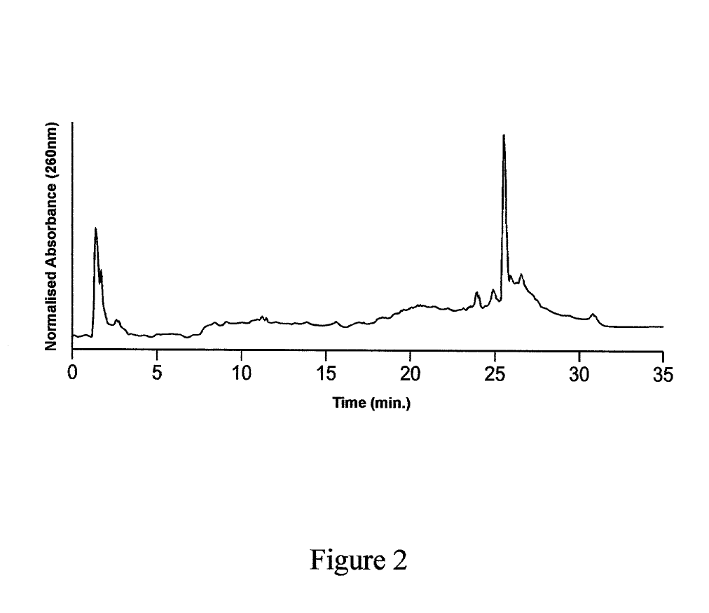

In order to characterize the density of immobilization, oligonucleotide synthesis was carried out as described above on GOPS-functionalized CPG, which has a well-defined surface area, in tandem with the oligonucleotide synthesis on the optical fibre substrates. The oligonucleotide-HEG conjugates were then cleaved from the surface of the CPG by exposure to concentrated ammonium hydroxide for approximately three hours, lyophilized and re-dissolved in 1.000 ml water. The sample was subsequently analyzed by anion-exchange HPLC. The chromatogram resulting from this synthesis is shown in FIG. 2. Quantitation of the cleaved HEG-dT₂₀ conjugates was achieved by co-injection with a known quantity of dT₂₀. The peak corresponding to a retention time of 25-26 minutes was thus attributed to dT₂₀. The distribution of species synthesized on the solid substrates may owe to the possible cross-linking within the underlying epoxysilane-linker layer and hence the number of epoxysilane moieties bound to the terminus of the released oligonucleotide-linker conjugate. The nucleic acid portion of the conjugate should therefore consist primarily of dT₂₀. This conclusion was made on the basis that the presence of incomplete oligonucleotide strands owing to poor synthon coupling would result in a series of resolved peaks of increasing area, which was not observed. The results of the HPLC analysis are shown in Table 2. The data show that oligonucleotide immobilization density was representative of a physical environment for the immobilized oligonucleotides in which the immobilized dT₂₀-HEG conjugates were separated by approximately 372.4 Å between adjacent strands, assuming uniform oligonucleotide distribution. Since the length of the dT₂₀-HEG conjugate is ca. 100 Å in length, this sample then represented the low-density case as described above, wherein there is, on average, very little chance of interactions between neighbouring strands that may affect hybridization.

TABLE 2
Density of Immobilization of dT₂₀-HEG Conjugates onto
GOPS-Functionalized Substrates as Determined by Anion-Exchange
High Performance Liquid Chromatography: Low density case.

| | Reaction Duration | Total Surface | Molecules | Average |
|---|---|---|---|---|
| Sam- | (DMT-HEG- | Area of CPG | dT₂₀-HEG | Radius per |
| ple | Substrate) (Hrs.) | Used (Å²) | Immobilized | Molecule (Å) |
| Low | 1 | 2.62 × 10¹⁹ | 2.41 × 10¹⁴ | 186.2 |
| Den- | | | | |
| sity | | | | |

### Example 2

### Control of Oligonucleotide Immobilization Density by the GOPS-HEG Method: Medium Density Case

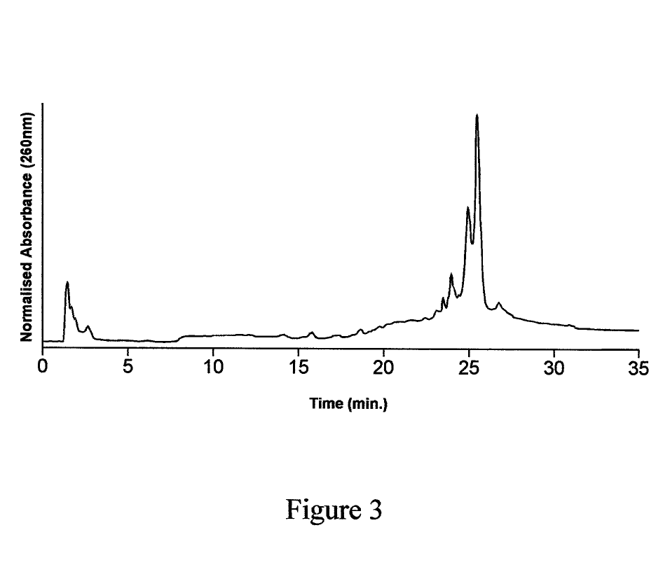

A second batch of substrates (CPG and fused silica optical fibres) was functionalized with GOPS as described above, and underwent the DMT-HEG coupling reaction using the same reaction mixture as described in example 1, for a duration of 4 hours. The dT₂₀-HEG conjugates were then cleaved from the surface of the CPG substrates as described in Example 1, lyophilized and redissolved in 1.000 mL water. This sample was then analyzed by AEHPLC. The resulting chromatogram is shown in FIG. 3. Quantitation of the cleaved HEG-dT₂₀ conjugates was again achieved by co-injection with a known quantity of dT₂₀. The peak corresponding to a retention time of 25-26 minutes was thus attributed to dT₂₀. The results of the HPLC analysis are shown in Table 3.

TABLE 3
Density of Immobilization of dT₂₀-HEG Conjugates onto
GOPS-Functionalized Substrates as Determined by Anion-Exchange
High Performance Liquid Chromatography: Medium density case.

| | Reaction Duration | Total Surface | Molecules | Average |
|---|---|---|---|---|
| Sam- | (DMT-HEG- | Area of CPG | dT₂₀-HEG | Radius per |
| ple | Substrate) (Hrs.) | Used (Å²) | Immobilized | Molecule (Å) |
| Med- | 4 | 2.62 × 10¹⁹ | 1.15 × 10¹⁵ | 85.3 |
| ium | | | | |
| Den- | | | | |
| sity | | | | |

These data indicate that oligonucleotide immobilization density was representative of a physical environment for the immobilized oligonucleotides in which the immobilized dT₂₀-HEG conjugates were separated by 170.6 Å between adjacent strands, which permit the onset of some interaction between neighbouring strands. Consequently, this sample is best denoted as medium density.

### Example 3

### Control of Oligonucleotide Immobilization Density by the GOPS-HEG Method: High Density Case

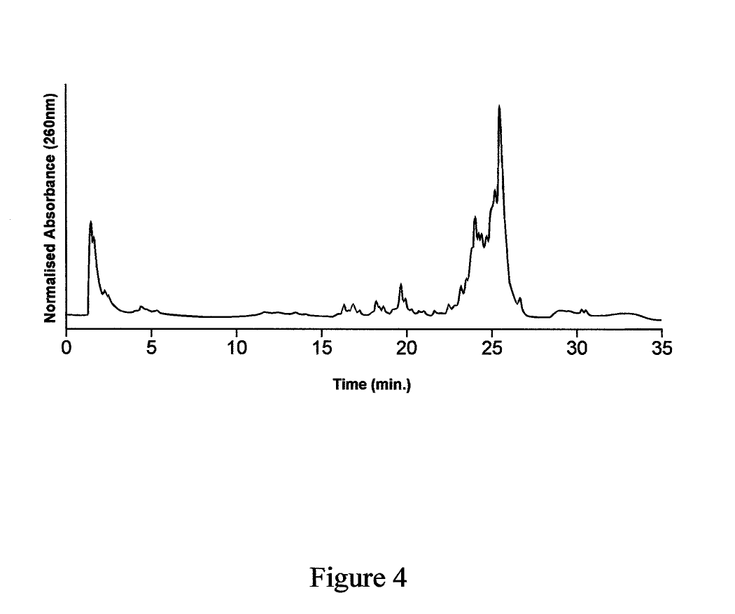

A third batch of substrates (CPG and fused silica optical fibres) were functionalized with GOPS as described above, and underwent the DMT-HEG coupling reaction using the same reaction mixture as described in examples 1 and 2, for a duration of 12 hours. The dT₂₀-HEG conjugates were then cleaved from the surface of the CPG substrates as described in Examples 1 and 2, lyophilized and redissolved in 1.000 mL water. This sample was then analyzed by AEHPLC. The resulting chromatogram is shown in FIG. 4. Quantitation of the cleaved HEG-dT₂₀ conjugates was again achieved by co-injection with a known quantity of dT₂₀. The peak corresponding to a retention time of 25-26 minutes was thus attributed to dT₂₀. The results of the HPLC analysis are shown in Table 4. These data indicate that oligonucleotide immobilization density was representative of a physical environment for the immobilized oligonucleotides in which the immobilized dT₂₀-HEG conjugates were separated by approximately 52.6 Å between adjacent strands. This close packing is much more likely to facilitate interactions between neighbouring strands than the lower packing densities. Consequently, this sample is most appropriately denoted as high density.

TABLE 4
Density of Immobilization of dT₂₀-HEG Conjugate onto
GOPS-Functionalized Substrates as Determined by Anion-Exchange
High Performance Liquid Chromatography

| | Reaction Duration | Total Surface | Molecules | Average |
|---|---|---|---|---|
| Sam- | (DMT-HEG- | Area of CPG | dT₂₀-HEG | Radius per |
| ple | Substrate) (Hrs.) | Used (Å²) | Immobilized | Molecule (Å) |
| High | 12 | 4.12 × 10¹⁹ | 1.90 × 10¹⁶ | 26.3 |
| Den- | | | | |
| sity | | | | |

### Example 4

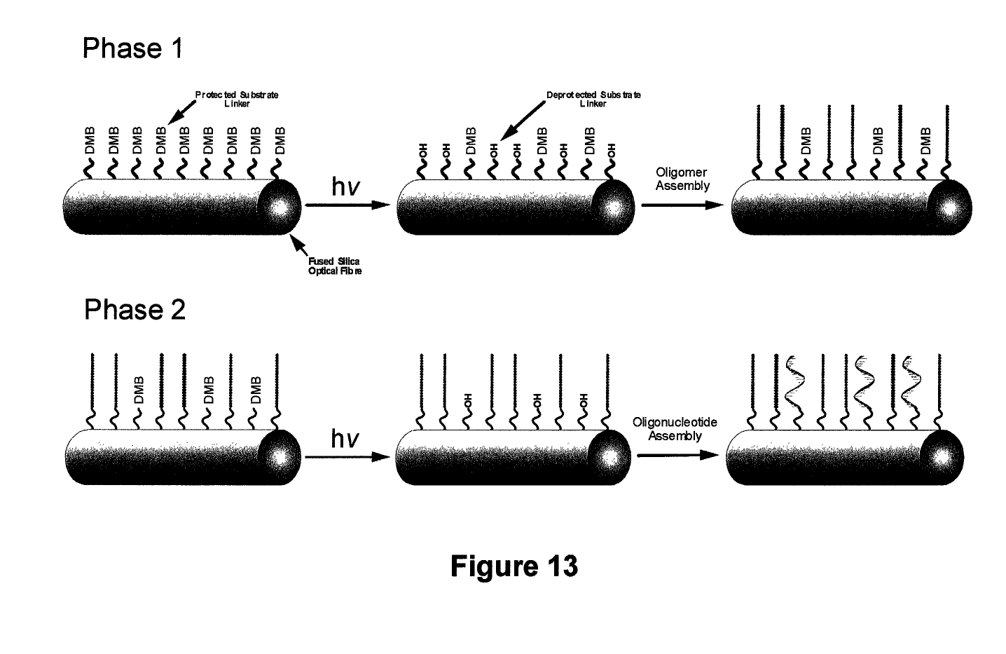

### Assembly of Mixed Films Containing Co-Immobilised Oligonucleotides and Oligomer Species (as illustrated in FIG. 13)

4.1: Preparation of DMB-HEG-OH

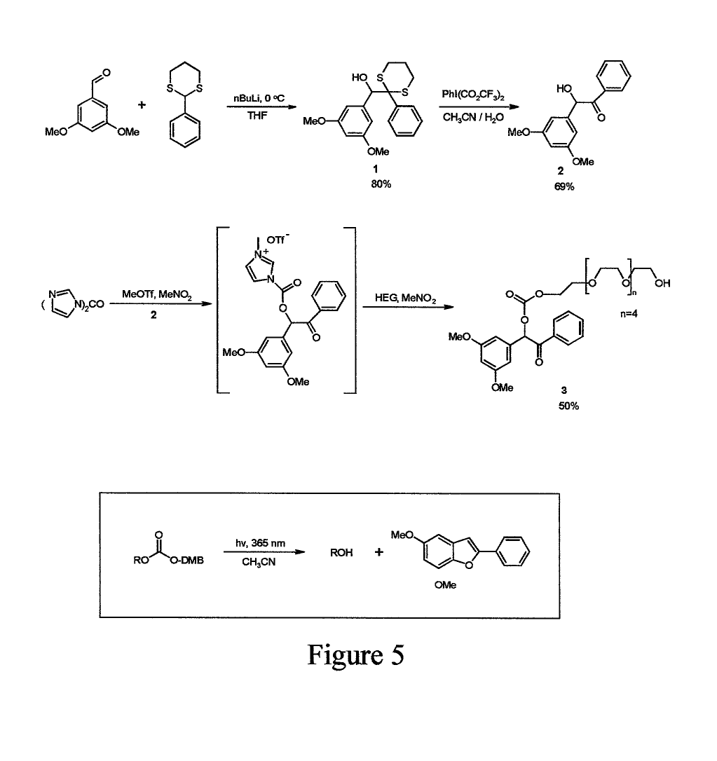

The synthetic route used for the preparation of the DMB-HEG-OH linker is shown graphically in FIG. 5 and described in detail in sections 4.1.1 to 4.1.3, which now follow.

4.1.1: Preparation of (3,5-Dimethoxy-phenyl)-(2-phenyl-[1,3]dithian-2-yl)-methanol¹ ¹Stowell, Michael H. B. et al. Tetrahedron Letters, 1996, vol. 37, No. 3, pp.307-310.

A solution of 2-phenyl-1,3-dithiane² (5.0 g, 0.0255 mol) in 85 ml of anhydrous tetrahydrofuran³ (THF) was cooled to 0° C.⁴ and 1.05 equivalents of nBuLi (10.7 ml, 2.5 M solution in hexane)⁵ was added dropwise via syringe with rapid stirring⁶, under an inert atmosphere of nitrogen. This solution was allowed to stir for 30 minutes at 0° C. and then 1.0 equivalents of 3,5-dimethoxybenzaldehyde⁷ (4.23 g, 0.0255 mol), dissolved in a minimal amount of anhydrous tetrahydrofuran³, was added dropwise over a period of 30 min. The solution was allowed to warm to room temperature⁸ and then stirred for an additional hour. The reaction is quenched by the addition of aqueous NH₄Cl. Tetrahydrofuran was removed in vacuo and the resultant slurry extracted with dichloromethane⁹ (100 ml). The organic phase was washed with 3×50 ml of distilled water¹⁰, brine (1×50 ml), dried (Na₂SO₄)¹¹, filtered¹² and concentrated in vacuo to yield crude material as a pale yellow oil. Column chromatography³ (silica gel, Hexane:Dichloromethane/7:3, R_{f}=0.0, followed by Dichloromethane, R_{f}=0.1)¹⁴ yielded 7.4 g (80%) of pure product¹⁵. δ_{H}(200 MHz; CDCl₃) 7.77-7.72 (2 H, m, aryl), 7.34-7.28 (3 H, m, aryl), 6.31 (1 H, t, J2.2, aryl), 6.00 (2 H, d, J2.2, aryl), 4.96 (1 H, bs, CH—OH), 3.59 (6 H, bs, CH₃O), 2.99 (1 H, bs, OH), 2.77-2.68 (4 H, m, (S—CH₂) and 2.03-1.92 (2 H, m, CH₂); m/z (EI) 362 (M⁺, 5%), 287 (25), 256 (75), 195 (100). ²White crystalline solid with strong odour. Handle in glove box. ³Moisture determination done by Coulometric Karl Fischer analysis: 55 ppm H₂O ⁴Exact temperature is not required. Ice-water bath is sufficient. ⁵Pale yellow liquid. FLAMMABLE upon exposure to moisture. Store at 0-5° C. Prior to use allow bottle to warm to room temperature. DO NOT remove sur-seal. All transfers should be done by syringe under an inert atmosphere. ⁶Accomplished by use of a magnetic stir bar and stir plate. Solution became yellow in colour. ⁷White crystalline solid. Handle in glove box. ⁸Requires approx. 2-2.5 hours. ⁹Distilled, not anhydrous, dichloromethane was sufficient. ¹⁰Extraction of the two phases was done using a separatory funnel. The organic phase (composed mostly of dichloromethane) was recovered as the heavier phase owing to the greater density of the organic solvent to that of water. ¹¹Anydrous sodium sulphate was used to remove water (2 or 3 spatula scoops). ¹²Gravity filtration using fluted filter paper. ¹³Flash chromatography is used. This involved the application of air pressure onto the solvent above the chromatography media in order to expedite the elution process. ¹⁴Once all other impurities (which have higher R_{f}'s) are eluted, the eluent is switched to dichloromethane (100%) to speed up the recovery of the desired product. ¹⁵Hygroscopic white foam.

4.1.2: Preparation of 2-(3,5-Dimethoxy-phenyl)-2-hydroxy-1-phenyl-ethanone (DMB-OH)^{1a,b} ^{1a}Stork, Gilbert; Zhao, Kang, Tetrahedron Letters, 1989, Vol. 30, No. 3, pp.287-290. ^{b}Photolysis occurs in standard laboratory light and product must be kept in complete darkness. Reaction must be performed in the dark or under red light (>630 nm wavelength).

Bis(trifluoroacetoxy)iodobenzene² (3.4 g, 0.0084 mol) was added at room temperature to a stirred solution of the dithiane benzoin adduct (2.45 g, 0.0067 mol) dissolved in 15 ml of acetonitrile:water/9:1.³ The reaction mixture was then stirred for 2.5 hours⁴. Saturated aqueous sodium bicarbonate (75 ml) was added followed by extraction of the mixture into dichloromethane⁵ (75 ml). The aqueous layer was further washed with dichloromethane (3×25 ml). The organic layer was then dried (Na₂SO₄)⁶, filtered⁷ and concentrated in vacuo to yield crude material as a pale yellow solid. Column chromatography⁸ (silica gel, dichloromethane, R_{f}=0.15)⁹ yielded 1.26 g (69%) of pure product¹⁰. δ_{H}(200 MHz; CDCl₃) 7.97-7.92 (2 H, m, aryl), 7.56-7.38 (3 H, m, aryl), 6.49 (2 H, d, J2.2, aryl), 6.37 (1 H, t, J2.2, aryl), 5.87 (1 H, d, J6.2, CH—OH), 4.54 (1 H, d, J6.2, OH) and (6 H, s, CH₃O); m/z (EI) 272 (M⁺, 34%), 167 (100), 139 (69), 105 (54), 77 (44). ²Pale yellow solid. Handle in glove box. Keep under an inert atmosphere until needed. ³HPLC grade acetonitrile and milli-Q water is used. ⁴Solution turned a pale orange-yellow. ⁵Distilled, not anhydrous, dichloromethane is sufficient. Extraction of the two phases was performed using a separatory funnel. Dichloromethane is the bottom phase as its density is greater. ⁶Anydrous sodium sulfate is used to remove any trace of water (2 or 3 spatula scoops). ⁷Gravity filtration using fluted filter paper. ⁸Flash chromatography is used. This involves the application of air pressure onto the solvent above the chromatography media in order to expedite the elution process. ⁹Once all other impurities (which have higher R_{f}'s) are eluted, the eluent is switched to dichloromethane:ether/1:1 to speed up the recovery of the desired product. ¹⁰Pale yellow solid.

4.1.3: Preparation of Carbonic acid 1-(3,5-dimethoxy-phenyl)-2-oxo-2-phenyl-ethyl ester 2-[2-(2-{2-[2-(2-hydroxy-ethoxy)-ethoxy]-ethoxy}-ethoxy)-ethoxy]-ethyl ester (DMB-HEG-OH)^{1a,b} ^{1a}Saha, Ashis K.; Schultz, Peter; Rapoport, Henry, J. Am. Chem. Soc., 1989, Vol. 111 , pp.4856-4859. ^{b}Photolysis occurs in standard laboratory light and product must be kept in complete darkness. Reaction must be done in the dark or under red light (>630 nm wavelength).

Methyl triflate² (3.85 g, 2.65 ml, 0.0234) was added dropwise via syringe to a solution of carbonyldiimidazole³ (1.9 g, 0.0117 mol) in anhydrous nitromethane⁴ (15 ml) at room temperature. The mixture was allowed to stir for 15 minutes⁵. A solution of 1,1-carbonylbis(3-methylimidazolium) triflate (prepared as above), was transferred into a suspension of DMB-OH (3.2 g, 0.0117 mol) in anhydrous nitromethane (15 ml)⁴. After 15 minutes, when CO₂ evolution ceased, a solution of hexaethylene glycol (6.62 g, 0.0234 mol) in anhydrous nitromethane⁴ (10 ml) was added via syringe. The reaction was quenched with water after 4 hours, and the mixture was extracted into dichloromethane⁶ (100 ml). The organic phase was washed with 5% aqueous Na₂CO₃ (2×50 ml), brine (2×50 ml), dried (Na₂SO₄)⁷, filtered8 and concentrated in vacuo to yield crude material as a yellow residue. Column chromatography⁹ (silica gel, dichloromethane, R_{f}=0.05)10 yielded 3.41 g (50%) of pure product¹¹. δ_{H}(200 MHz; CDCl₃) 7.98-7.93 (2 H, m, aryl), 7.55-7.39 (3 H, m, aryl), 6.66 (1 H, s, CHO), 6.62 (2 H, d, J 2.2, aryl), 6.43 (1 H, t, J 2.2, aryl), 4.34 (2 H, t, J 4.0, OCH₂), 3.78 (6 H, s, CH₃O) and 3.67 (22 H, s, CH₂); m/z (EI) 583 (M⁺, 1%), 298 (57), 255 (50), 149 (65), 105 (94), 89 (100), 77 (45). ²Colourless liquid. Handle in glove box ³White solid. Handle in glove box ⁴Moisture determination done by Coulometric Karl Fischer analysis: 69 ppm H₂O ⁵The solution turned yellow. The reaction is very fast and the 1.1′-carbonylbis(3-methylimidazolium) triflate (quantitative yield assumed) generated is used directly for acyl activation. ⁶Distilled, not anhydrous, dichloromethane is sufficient. Extraction of the two phases was done using a separatory funnel. The organic phase composed mostly of dichloromethane was recovered as the heavier phase owing to the greater density of the organic solvent to that of water. ⁷Anydrous sodium sulphate was used to remove water (2 or 3 spatula scoops). ⁸Gravity filtration using fluted filter paper. ⁹Flash chromatography was used. This involved the application of air pressure onto the solvent above the chromatography media in order to expedite the elution process. ¹⁰Once all other impurities (which had higher R_{f}'s) were eluted, the eluent was gradually increased to dichloromethane:methanol/9.5:0.5 to speed up the recovery of the desired product. ¹¹Pale yellow oil.

4.2: Preparation of DMT-EG-Phosphonamidite (Diisopropyl-phosphoramidous acid 2-[bis-{4-methoxy-phenyl}-phenyl-methoxy]-ethyl ester methyl ester):

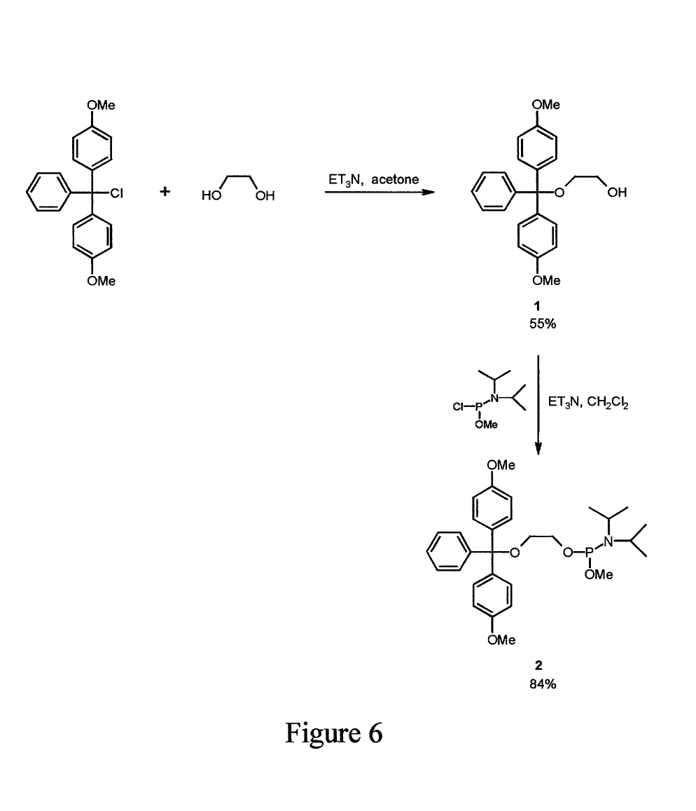

The synthetic route used for the preparation of the DMT-EG-Phosphonamidite synthon is shown graphically in FIG. 6 and described in detail in sections 4.2.1 and 4.2.2, which now follow.

4.2.1: Preparation of 2-[Bis-(4-methoxy-phenyl)-phenyl-methoxy]-ethanol (DMT-EG)¹ ¹Compound is temperature and acid sensitive. Do not heat above 35° C.

To a solution of ethylene glycol² (5.0 g, 0.081 mol) in 15 ml of anhydrous acetone was added triethylamine³ (8.15 g, 11.23 ml, 0.081 mol). After stirring for 10 min, a solution of 4,4-dimethoxytrityl chloride⁴ (13.65 g, 0.040 mol) in 145 ml of anhydrous acetone was added dropwise over a period of 6 h⁵. The reaction mixture was then allowed to stir overnight. The resulting mixture was filtered⁶ and then concentrated in vacuo. The resulting oily residue was extracted into dichloromethane⁷ (150 ml), washed with water (3×75 ml), dried (Na₂SO₄)⁸, filtered⁹ and concentrated in vacuo to yield crude material as an orange oil. Column chromatography¹⁰ (silica gel, Dichloromethane:Ether:Et₃N/96:2:2, R_{f}=0.2 yielded 8.1 g (55%) of pure product¹¹. δ_{H}(200 MHz; CDCl₃) 7.47-7.29 (9 H, m, aryl), 6.86-6.81 (4 H, m, aryl), 3.80 (6 H, s, CH₃O), 3.80-3.73 (2 H, m, CH₂) and 3.26 (2 H, t, J4.4,CH₂). ²Clear liquid. Handle in glove box. ³Clear liquid. Handle in glove box. ⁴Orange solid. Handle in glove box. Temperature and acid sensitive reagent. ⁵After complete addition of the 4,4-dimethoxytrityl chloride solution the reaction mixture was orange in colour with the presence of white precipitate (triethylamine salt). ⁶Under vacuum using a scinter glass funnel. Solution was orange. ⁷Distilled, not anhydrous, dichloromethane is sufficient. Extraction of the two phases was done using a separatory funnel. The organic phase composed mostly of dichloromethane was recovered as the heavier phase owing to the greater density of the organic solvent to that of water. ⁸Anydrous sodium sulphate was used to remove water (2 or 3 spatula scoops). ⁹Gravity filtration using fluted filter paper. ¹⁰Flash chromatography was used. This involved the application of air pressure onto the solvent above the chromatography media in order to expedite the elution process. ¹¹Pale yellow oil.

4.2.2: Preparation of Diisopropyl-phosphoramidous acid 2-[bis-(4-methoxy-phenyl)-phenyl-methoxy]-ethyl ester methyl ester (DMT-EG-Phosphonamidite)¹ ¹Compound is temperature and acid sensitive. Do not heat above 35° C.

To a solution of DMT-EG (2.0 g, 0.0055 mol) in 15 ml of anhydrous dichloromethane² was added triethylamine³ (1.39 g, 1.91 ml, 0.0137 mol). After stirring for 15 min, N,N-diisopropylmethylphosphonamidite chloride⁴ (1.19 g, 0.006 mol) was added dropwise over a period of 1.5 h⁵. The reaction mixture was then allowed to stir overnight. The resulting mixture was then concentrated in vacuo to give an oily residue. Column chromatography⁶ (silica gel, Ether:Et₃N/98:2, R_{f}=0.8 yielded 2.42 g (84%) of pure product⁷. δ_{H}(200 MHz; CDCl₃) 7.52-7.29 (9 H, m, aryl), 6.85-6.76 (4 H, m, aryl), 3.79 (6 H, s, CH₃O-aryl), 3.79-3.61 (4 H, m, CH₂), 3.43 (3 H, d, J 12.8, OCH₃), 3.28-3.20 (2 H, m CH) and 1.20 (12 H, d, J7.0,CH₃). ²Moisture determination done by Coulometric Karl Fischer analysis: 0.0 ppm H₂O. ³Clear liquid. Handle in glove box. ⁴Clear liquid. Violently hydrolyses upon exposure to moisture. Use only in glove box. ⁵Presence of white precipitate (triethylamine salt) results near the final addition of the chloride. ⁶Flash chromatography was used. This involved the application of air pressure onto the solvent above the chromatography media in order to expedite the elution process. ⁷Hydroscopic white foam/pale yellow oil.

4.3: Sensor Preparation

500 fibres pieces were prepared as per the method detailed in example 1.3. The fibres, in addition to 4 g of CPG, were cleaned and functionalised with GOPS as per the methods described in examples 1.4 and 1.5, respectively.

4.3.1: Acid Catalyzed Epoxide Hydrolysis of GOPS Functionalized Substrates.

The GOPS functionalized substrates (500 fibres and 4 g CPG) were suspended in 400 ml of a solution of 10% dichloroacetic acid in water. Hydrolysis was done at room temperature for 2 hours. The aqueous solution was decanted and the substrates were successively washed with two 200 ml portions of each of water, methanol, dichloromethane and diethyl ether. The substrates were dried and stored in-vacuo at 55° C. until required.

4.3.2: Activation of Hydrolyzed GOPS Functionalized Substrates by Treatment with Methanesulfonyl Chloride.

The hydrolysed GOPS functionalised substrates were immersed in a solution of methanesulfonyl chloride in acetonitrile (1% v/v, 10 ml/100 mg CPG, water content of 19.4 ppm). The reaction was permitted to proceed at room temperature for 1 hour under an anhydrous atmosphere with gentle agitation. The solution of methanesulfonyl chloride was then decanted and the substrates recovered and rinsed once with 10 mL of anhydrous acetonitrile followed by drying in-vacuo for 2 hours.

4.3.3: Linkage of DMB-HEG onto Mesylated Substrates

DMB-HEG (7.0 g) that had been dried by extended storage in-vacuo was dissolved in anhydrous acetonitrile to make a solution of 8.16×10⁻²M DMB-HEG. Mesylated substrates were separated into batches, each batch containing both optical fibres (20) and 100 mg of CPG. 10 mL of acetonitrile and 10 mL of the DMB-HEG solution was added to the substrates. The reaction was permitted to proceed in darkness, under an anhydrous atmosphere at room temperature with gentle agitation for a duration of 30 minutes. The reaction mixture was then decanted and the substrates were recovered and washed with three 20 ml portions of anhydrous acetonitrile to quench the coupling reaction and remove non-specifically adsorbed reactants. The DMB-protected HEG-functionalized substrates were kept in the dark and in-vacuo until required.

4.3.4: Photodeprotection of DMB-HEG Functionalized Substrates.

DMB-HEG functionalized substrates were divided into three batches, two of which were treated to photodepreotection prior to solid-phase assembly of oligomers for times of 3 minutes and 1 hour. Photodeprotection was done using a General Electric 85W H85A3 UV mercury lab-arc lamp powered by a MLA-85 Power Supply (Gates, Franklin Park, LI, N.Y.) operated at full power. Substrates were placed in a transparent glass vessel (20 ml volume) to which 10 ml acetonitrile was added. The photodeprotection reaction vessel containing substrates and solvent were rotated at a constant speed (60 rpm) and irradiated at a distance of 60 centimeters relative to the mercury lamp. The photolysis products formed on release of the DMB moiety are shown in the box at the bottom of FIG. 5. Following photodeprotection, the substrates were recovered and washed with 10 mL of acetonitrile. The three substrates were kept in darkness and in-vacuo until required.

4.3.5: Solid Phase Phosphoramidite Synthesis of Polyelectrolyte on to Photodeprotected Substrates

Solid phase oligonucleotide synthesis was done as described in example 1.8. Ethylene glycol based oligomers or polythymidylic acid icosanucleotides were assembled onto the optical fibre and CPG substrates functionalised with DMB-HEG linker molecules, with varying portions of the linker molecules photodeprotected so as to permit the assembly of mixed films containing non-nucleic acid oligomers (by use of the ethylene glycol-based synthon described in 4.2 and the standard commercially available N-benzoyl-2′-deoxycytidine phosphoramidite synthon) and polythymidylic acid icosanucleotides. Two N-benzoyl-protected cytidine residues were incorporated at both the 5′ and 3′ ends of the polyethylene glycol-based oligomers in order to permit detection of the synthesis products by absorbance at 260 mn during AE-HPLC analysis determinations of the density and synthesis quality of the polyethylene glycol oligomers.

Ethyleneglycolphosphate (EGp) Oligomer: CCE EEE EEE EEE EEE EEE CC, where E is an Ethylene Glycol Moiety and C an N-benzoyl Protected Cytidine Moiety.

Note: The benzoyl protecting group was not removed from the cytidine residues on the fibre surface so as to block interaction of the nucleotides with nucleic acids introduced into the system.

4.3.6: Solid Phase Phosphoramidite Synthesis of Polythymidylic acid icosanucleotides onto Photodeprotected Substrates.

All substrates were further photodeprotected using the method described in 4.3.4 for a time of 1 hour prior to assembly of polythymidylic acid icosanucleotides.

4.3.7: Characterisation of Nucleic Acid and Mixed Film Composition.

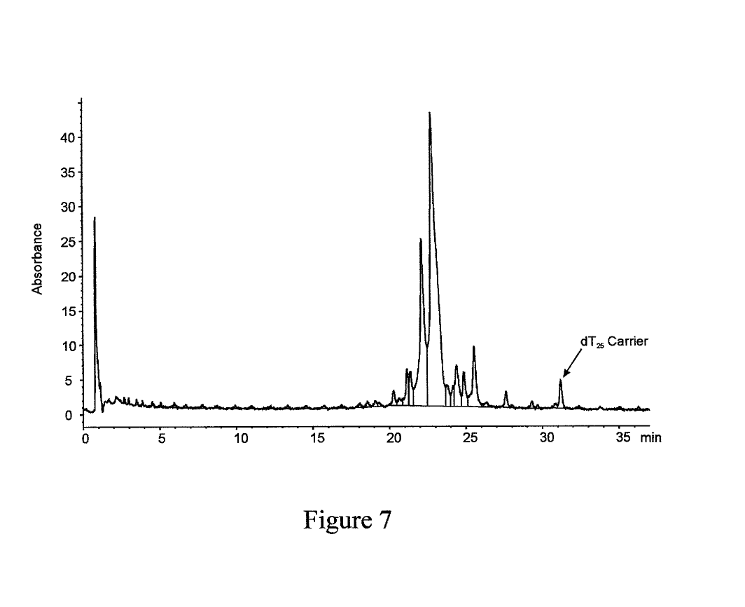

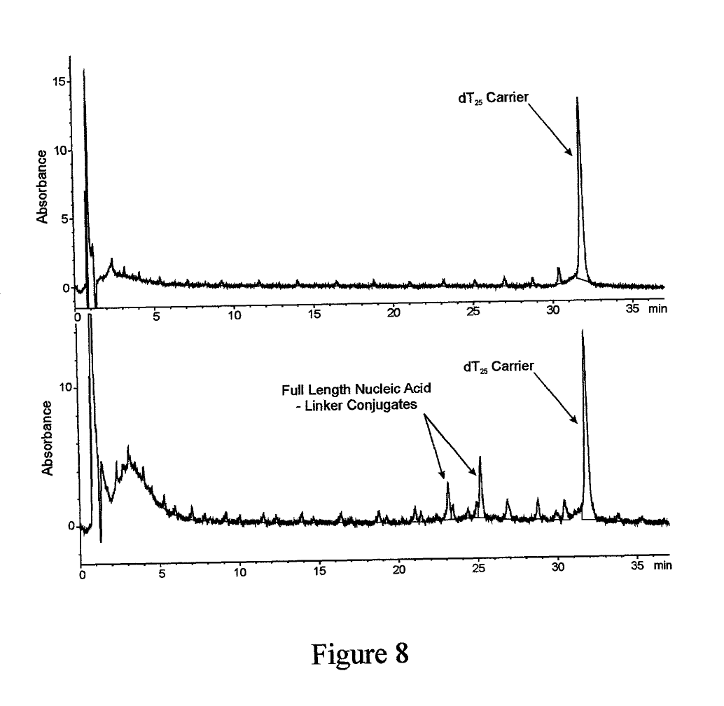

Cleavage of oligonucleotide and polyethylene glycol oligomers assembled onto CPG substrates and analysis of oligomer density and synthesis fidelity was done as per the methods described in examples 1.9 and 1.10, respectively. Representative AE-HPLC chromatograms of the products and carrier recovered from CPG substrates for the assembly of the ethyleneglycolphosphate oligomers and subsequent assembly of dT₂₀ to create a mixed film of immobilised oligomers and films containing only oligonucleotide-linker conjugates are shown in FIGS. 7 and 8.

4.4: Discussion.

The assembled films composed of 100% nucleic acid—linker conjugates by the methods recited in this example were observed to provided similar AE-HPLC chromatograms to those observed in examples 1-3, as shown in FIG. 7. The distribution of products owing to the possible cross-linking within the underlying epoxysilane-linker layer and hence the number of epoxysilane moieties bound to the terminus of the released oligonucleotide-linker conjugate was observed to be of lower magnitude. A reduction in epoxide cross-linking in the epoxysilane layer may have been the result of the hydrolysis step done prior to the mesylate-mediated coupling of the linker to the silanised substrate. Sensors created based on this chemistry were all of consistently high packing density. The mean centre-to-centre strand separation distance for films consisting only of oligonucleotide-linker conjugates was found to be ca. 20 Å.

Films of mixed ethyleneglycolphosphate based oligomer-linker conjugates and oligonucleotide-linker conjugates were prepared by a two-phase chemical assembly protocol. In the first phase, limited photodeprotection was done followed by assembly of the ethyleneglycolphosphate based oligomer. The initial photolysis procedure served to remove the terminal DMB protecting group from a portion of the immobilised linker molecules so as to permit oligomer assembly to occur from those sights. Following assembly of the first oligomer, the substrates were capped to prevent further synthon coupling onto the existing oligomers, and then treated to extended photolysis so as to quantitatively remove the remaining DMB groups from the remaining protected substrate linkers. Assembly of the oligonucleotide onto those sites was then done in the second phase of the procedure to yield an immobilised film of mixed oligomer composition. AE-HPLC analysis of the synthesis products assembled onto CPG was done following the addition of a carrier oligonucleotide to the support and cleavage of the oligomers from the support by amminolysis. The assembled film of mixed oligomers was found to have a mean centre-to-centre strand separation distance of ca. 50 Å, with a composition of 5±4 mole percent of immobilised oligonucleotide-linker conjugate relative to the ethyleneglycolphosphate based oligomers. As shown in the top chromatogram of FIG. 8, the fidelity of synthesis of the ethyleneglycolphosphate-based oligomer was poor. As the distribution of oligomer products was not consistent with poor synthon coupling efficiency, it was speculated that the formation of distributed length products was the result of ongoing loss of the photolabile DMB protecting group from protected linker molecules. The most probably cause of this likely owed to leakage of light into the synthesis column during oligomer assembly. For the purposes of these experiments, oligomers consisting of more than 15 coupled synthon units (cytidine-phosphoramidite or ethylene glycol-phosphoramidite) were used in the calculations of the quantity of immobilised strands and strand packing density.

### Example 5

### Comparison of Nucleic Acid Hybridization in Interfacial and Bulk Solution Environments: Determination of Nucleic Acid Hybridization Thermodynamic Parameters in Bulk Solution

Thermal denaturation profiles were obtained for oligonucleotides hybridized in bulk solution, in an effort to determine some of the trends in the thermodynamics of hybridization as it occurs with dissolved oligonucleotides in bulk solution. Initial experiments consisted of an examination of the relationship between the observed thermal denaturation temperature, Tₘ, and the ionic strength of the hybridization solution. In the these experiments dT₂₀ (0.62 μM) was hybridized with one of the following oligonucleotides in a 1:1 molar ratio: dA₂₀, d(A₉GA₁₀), d(A₉G₂A₉), d(A₁₈G₂), d(G₂A₁₆G₂) or d(G₅A₂₀G₅). Hybridization was carried out in a solution of PBS (1 M NaCl, 50 mM NaH₂PO₄, 50 mM Na₂HPO₄) buffer diluted by a factor of 1.0, 0.75, 0.5, 0.3 or 0.1. In order to determine thermodynamic parameters for the thermal denaturation process, the raw absorbance data was used to compute values of the total fraction of ssDNA present in the system at any of the measured temperature points. In so doing, it was assumed that the denaturation process consisted of a two-state, all-or-nothing transition between the completely hybridized and completely denatured states for any given duplex. The fraction of ssDNA, ƒₛₛ, was then computed by means of the following equation:

$${f}_{ss} = \frac{A ( T ) - {A}_{ss} ( T )}{{A}_{ds} ( T ) - {A}_{ss} ( T )} \qquad ( 1 )$$

where A(T) represents the total absorbance of the system at any temperature, T and Aₛₛ(T) and A_{ds}(T) represent the absorbance due to fully denatured and fully hybridized DNA, respectively. The parameters Aₛₛ(T) and A_{ds}(T) were obtained by extrapolating the fitted linear baseline data in the lower and higher temperature regions of the profile over the entire temperature range used. In these experiments, where equimolar concentrations of complementary oligonucleotides were used, the value of Tₘ was computed by determining the temperature at which the value of ƒₛ was equal to 0.5.

This method of analysis was repeated for all thermal denaturation experiments conducted with oligonucleotides in bulk solution. The values of Tₘ obtained as a function of hybridization buffer ionic strength are shown in Table 5 for all oligonucleotide hybrids used. These results illustrate that the presence of base-pair mismatches has the potential to reduce the observed Tₘ value of the duplex. Furthermore, the deviation in Tₘ for a duplex that contains base-pair mismatches from that of the fully complementary duplex is a function of the ionic strength of the hybridization solution, the number of base-pair mismatches and their positions within the duplex. Table 5 illustrates that the difference in Tₘ between the fully complementary dsDNA sequence dA₂₀:dT₂₀ and that containing a centrally located single base-pair mismatch (SBPM) can be as large as 6° C. Similarly, the data also show differences in Tₘ as large as 10.1° C. between the fully complementary dsDNA sequence relative to that which contained two centrally located base-pair mismatches. However, when the two base-pair mismatches were located at a terminus of the double helix, the difference in Tₘ became insignificant.

TABLE 5
Tₘ (° C.) Values Obtained for dT₂₀ Hybridized with Various Oligonucleotides, in
Hybridization buffers of Various Ionic Strengths.

| | Thermal Denaturation Temperature, Tₘ (° C.) for dT₂₀ Hybridized with | | | | | |
|---|---|---|---|---|---|---|
| | (Total [dsDNA] = 0.62 μM, equimolar amounts of ssDNA) | | | | | |
| | dA₂₀ | d(A₉GA₁₀) | d(A₉G₂A₉) | d(A₁₈G₂) | d(G₂A₁₆G₂) | d(G₅A₂₀G₅) |
| [NaCl] (M) | | | | | | |
| 1.0 | 57.6 ± 0.4 | 52.4 ± 0.5 | 48.7 ± 0.6 | 56.9 ± 0.4 | 53.5 ± 0.7 | 58.9 ± 0.7 |
| 0.75 | 55.7 ± 0.5 | 51.7 ± 0.4 | 46.6 ± 0.3 | 55.1 ± 0.5 | 50.8 ± 0.7 | 56.9 ± 0.7 |
| 0.50 | 53.5 ± 0.4 | 48.3 ± 0.4 | 44.2 ± 0.3 | 52.3 ± 0.4 | 49.2 ± 0.5 | 54.5 ± 0.6 |
| 0.30 | 50.6 ± 0.6 | 44.5 ± 0.5 | 41.0 ± 0.4 | 49.8 ± 0.4 | 44.9 ± 0.6 | 50.5 ± 0.5 |
| 0.1 | 43.3 ± 0.6 | 37.4 ± 0.6 | 33.2 ± 0.5 | 43.0 ± 0.5 | 37.0 ± 0.6 | 43.2 ± 0.7 |
| ∂Tₘ/∂log[Na⁺] | | | | | | |
| (° C.) | 14.2 ± 0.3 | 15.6 ± 0.7 | 15.4 ± 0.3 | 13.8 ± 0.3 | 16.3 ± 0.7 | 15.8 ± 0.3 |
| R² | 0.998 | 0.994 | 0.999 | 0.999 | 0.995 | 0.999 |

The difference in Tₘ for a dsDNA sequence containing a centrally located SBPM relative to that of the fully complementary duplex is a function of the ionic strength of the hybridization solution. The practical implication of this behaviour is that the selectivity of a hybridization assay can be controlled more with greater stringency at lower ionic strengths than at higher ionic strengths, where the differences in Tₘ between the complementary sequences and those containing an SBPM are larger.

The difference in Tₘ between a fully complementary dsDNA sequence and that containing an SBPM is also a function of the total strand concentration. The strand concentration dependence of the Tₘ is described by the equation:

$$\frac{1}{{T}_{m}} = \frac{R}{Δ {H}^{o}} ln {C}_{T} + \frac{Δ {S}^{o} - R ln 4}{Δ {H}^{o}} \qquad ( 2 )$$

where R is the gas constant, C_{T} is the total strand concentration, and ΔH° and ΔS° are the standard enthalpy and standard entropy changes, respectively. It should be noted that the enthalpic and entropic changes predicted by this equation are average values only, since the equation assumes them both to be temperature independent, which has been recently refuted by Breslauer [25]. Based on equation (2), a small difference in the sensitivity of Tₘ to strand concentration between these two dsDNA sequences would be expected on the basis that there should be a difference in the enthalpy change accompanying the denaturation event. This difference can be seen when computing the van't Hoff enthalpy changes from the normalized thermal denaturation data, which will be discussed in more detail below.

The van't Hoff enthalpy change is the enthalpy change accompanying the denaturation event, computed under the assumption that denaturation is a two-state transition. The van't Hoff enthalpy change at Tₘ is computed from the normalized thermal denaturation data by means of equation (3):

$$Δ {H}_{VH , {T}_{m}} = - 6 {{RT}_{m} ( \frac{\partial {F}_{ss}}{\partial T} )}_{T = {T}_{m}} \qquad ( 3 )$$

When values of ΔH_{VH} are computed for a given duplex in hybridization buffer at various ionic strengths, values of ΔH_{VH} as a function of temperature are obtained. Recently, Breslauer [18] reported that the enthalpy change accompanying denaturation was in fact a function of temperature as a result of a small change in the heat capacity of the system as a result of denaturation, which is contrary to assumptions made hitherto in studies of oligonucleotide hybridization thermodynamics [26]. It is therefore possible to use values of ΔH_{VH} obtained at Tₘ in hybridization buffers of different ionic strengths to compute values of ΔH° at a standard reference temperature, in order to establish a basis of comparison for the relative stability of two different sequences. In general, the enthalpy change for a given process is a function of temperature according to the following relation [27]:

$$Δ {H}_{T}^{o} = Δ {H}_{{T}_{ref}}^{o} + {\int }_{{T}_{ref}}^{T} Δ {C}_{p} ⅆ T \qquad ( 4 )$$

Assuming that ΔCₚ is independent of temperature, and using a value for T_{ref} of 40° C., then the above equation can be integrated and rearranged to yield:

ΔH°_{40° C.}=ΔH°_{T}_{ₘ}−ΔCₚ(Tₘ−40° C.) (5)

The value of ΔCₚ may be obtained by computing the slope of a plot of ΔH_{VH} versus Tₘ from denaturation experiments in hybridization buffers of different ionic strengths. The values of ΔH_{VH} at Tₘ and ΔH° corrected to 40° C. for dA₂₀:dT₂₀ and d(A₉GA₁₀):dT₂₀ in hybridization buffers of various ionic strengths are shown below in Table 6. These values correspond to a value of ΔCₚ of 112±3 cal.deg⁻¹.mol_{bp} for dA₂₀:dT₂₀, which is in good agreement with the value for the polymeric duplex poly(dA):poly(dT) presented by Breslauer (101.7±24 cal.deg⁻¹.mol_{bp}).

These data show that there is a difference in the enthalpy change of the denaturation event between the fully-complementary dsDNA sequence and that containing a centrally located SBPM. This difference is due to the imperfect Watson-Crick base-pairing and the resulting bulge in the double-helix at the SBPM site, which in turn affects the hydrogen bond strength at nearest-neighbour sites as a result of stretching of the base-pair interactions brought about by the bulge. This difference is also consistent with the observations of deviations in Tₘ in dsDNA sequences containing an SBPM relative to the fully complementary sequences.

TABLE 6
van't Hoff Enthalpy Changes at Tₘ and Corrected to 40° C.
for 0.62 μM Solutions of dA₂₀:dT₂₀ and d(A₉GA₁₀):dT₂₀ in
Hybridization Buffers of Various Ionic Strengths.

| | dA₂₀:dT₂₀ | | | d(A₉GA₁₀):dT₂₀ | | |
|---|---|---|---|---|---|---|
| | | ΔH_{VH} | ΔH° | | ΔH_{VH} | ΔH° |
| [NaCl] | Tₘ | (Tₘ) | (40° C.) | Tₘ | (Tₘ) | (40° C.) |
| (M) | (°C.) | (kcal/mol) | (kcal/mol) | (°C.) | (kcal/mol) | (kcal/mol) |
| 1.0 | 57.8 | 168 | 128 | 52.4 | 137 | 105 |
| 0.5 | 53.9 | 162 | 131 | 48.0 | 138 | 114 |
| 0.3 | 51.2 | 153 | 138 | 44.0 | 118 | 106 |
| | | Mean | 129 ± 2 | | Mean | 108 ± 5 |

### Example 6

### Comparison of Nucleic Acid Hybridization in Interfacial and Bulk Solution Environments: Determination of Nucleic Acid Hybridization Thermodynamic Parameters in Interfacial Environments

Experiments illustrating the relationship between oligonucleotide immobilization density and the thermodynamic selectivity of nucleic acid hybridizations occurring at solid-liquid interfaces were done using a fibre-optic nucleic acid biosensor based on total internal reflection fluorescence, wherein probe oligonucleotides were covalently bound to the surface of fused silica optical fibres via flexible polyether linkers [28]. Thermal denaturation profiles were obtained for oligonucleotides covalently immobilized to the surface of fused silica optical fibres in an effort to determine if trends observed for hybridization experiments carried out in bulk solution could be extrapolated to describe the behaviour of DNA hybridization at an interface. Since many nucleic acid biosensor schemes involve hybridization of oligonucleotides immobilized to a solid surface, it is of obvious importance to establish trends in the hybridization thermodynamics for such systems in order to address issues of sensitivity and selectivity.

Studies of hybridization thermodynamics of fully complementary hybrids and those containing a centrally located SBPM were done using dA20-5′-fluorescein and d(A₉GA₁₀)-⁵′-fluorescein, respectively, at a concentration of 10⁻⁷ M. Thermal denaturation experiments were done using the fibre optic biosensor instrument described elsewhere [28]. Excitation radiation was delivered to the nucleic acid membrane by means of coupling a beam from an Argon ion laser (λₘₐₓ=488 nm) into the optical fibre. The fluorescent emission was coupled back into the optical fibre and collected at a wavelength of 542 nm. The temperature was ramped in these experiments over the range 25-100° C. at a rate of 0.3° C.min⁻¹. Complementary oligonucleotides were introduced in hybridization buffers of various ionic strengths (0.1, 0.3, 0.5 or 1 M NaCl) in an effort to establish the trends in interfacial hybridization thermodynamics as they relate to the ionic strength of the hybridization solution.

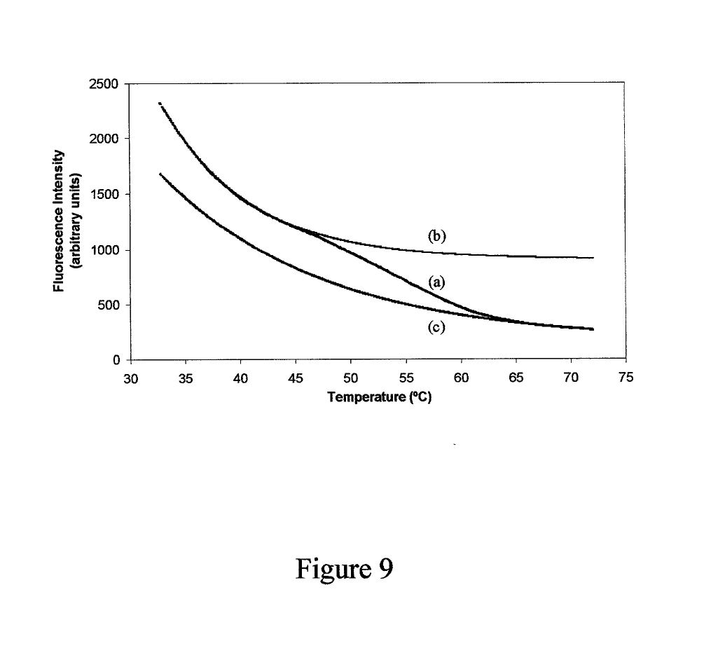

An example of the raw data obtained from the thermal denaturation profiles measured at the surface of the optical fibre biosensors with medium packing density of immobilized dT₂₀ is shown in FIG. 9. Again, the data was analyzed under the assumption that the denaturation took place as a two-state transition. The upper and lower baselines were used to extrapolate the thermal fluorescence decay. It was assumed that full hybridization occurred and that the oligonucleotides were fully denatured in the high temperature regime. Mono-exponential decay profiles were fitted to the baseline data in the case of fluorescence measurements since this type of profile was found to accurately describe the thermal fluorescence decay obtained when a fused silica fibre treated only with trimethylchlorosilane (TMS-Cl) was exposed to a solution of dA₂₀ and dA₂₀-fluorescein described above, and subjected to the same temperature ramping conditions (data not shown). Conversion of the raw data to a normalized thermal denaturation profile consisting of the normalized fraction of ssDNA present as a function of temperature was then achieved by use of equation (1) and treatment analogous to that used for the raw absorbance data.

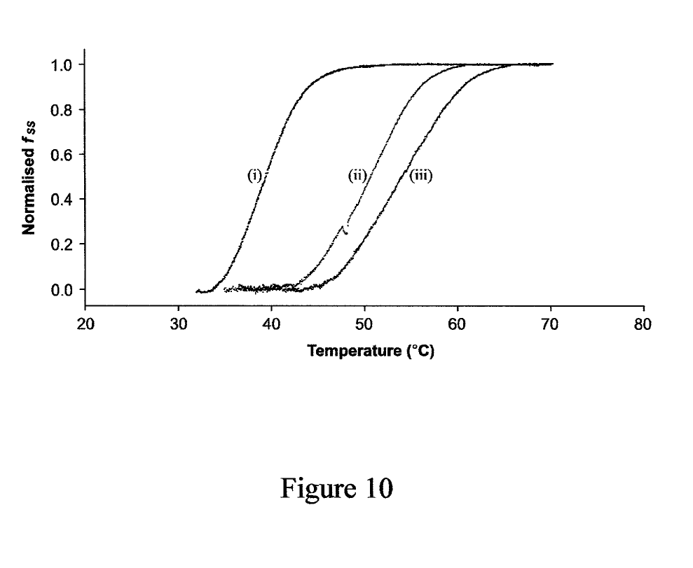

The normalized thermal denaturation profiles obtained using the optical fibre biosensor with low oligonucleotide packing density in hybridization buffers of different ionic strengths and using dA₂₀-fluorescein as the complementary material are shown in FIG. 10. The Tₘ data observed as a function of ionic strength for the low, medium and high packing densities and using dA₂₀-fluorescein as the complementary material are shown in Table 7.

TABLE 7
Observed Tₘ (° C.) values for Optical Fibre Biosensors
with Low, Medium and High Oligonucleotide Packing Densities Using
Hybridization Buffers of Various Ionic Strengths and dA₂₀-
fluorescein as the Complementary Material

| | Low | Medium | High |
|---|---|---|---|
| | Packing Density | Packing Density | Packing Density |
| [NaCl] (M) | Tₘ (° C.) | Tₘ (° C.) | Tₘ (° C.) |
| 0.1 | 39.5 ± 0.2 | 41.6 ± 0.2 | 32.3 ± 0.2 |
| 0.5 | 50.7 ± 0.2 | 48.0 ± 0.2 | 43.5 ± 0.2 |
| 1.0 | 54.9 ± 0.2 | 53.1 ± 0.2 | 46.4 ± 0.2 |
| ∂Tₘ/∂log [Na⁺] | 15.5 ± 0.5 | 11 ± 2 | 14 ± 1 |
| (° C.) | | | |

These data illustrate the effect of packing density on the thermodynamics of hybridization. It appeared that the high packing density facilitated some destabilization of the hybridized immobilized oligonucleotides as evidenced by the Tₘ values which were consistently lower than those observed with the low packing density and medium packing density optical fibre biosensors. Additionally, the sensitivity of Tₘ to salt concentration in the hybridization buffer appeared to be fairly consistent with observations made in bulk solution, and the three values obtained agree within experimental uncertainty at the 95% confidence level. This supports the notion that there is no significant difference in the ion environments within the nucleic acid membranes brought about as a function of oligonucleotide packing density, as predicted by a theoretical model described elsewhere [28]. It may be that the differences in Tₘ observed with the optical fibre biosensor with high oligonucleotide packing density relative to those with the low and medium packing densities is a result of greater interaction between neighbouring strands, whereby the interactions interfere with the hydrogen bonding between complementary base pairs and reduce the overall stability of the hydrogen bonds. These interactions may also reduce the number of immobilized oligonucleotides that are available for hybridization, similar to what has been reported by Southern [16].

In order to establish trends in the hybridization energetics which govern selectivity, thermal denaturation experiments identical to those described above were performed using the low, medium and high packing density optical fibre biosensors and d(A₉GA₁₀)-fluorescein as the complementary material. The observed Tₘ values in those experiments are listed below in Table 7.

Examination of the data in Table 7 and Table 8 shows that for the optical fibre biosensors with low and medium oligonucleotide packing density, the deviations in Tₘ caused by the presence of a centrally located SBPM were larger when hybridization occurred in solutions of lower ionic strength, relative to those observed in experiments done in hybridization buffers with higher ionic strength. This observation, which is consistent with observations made in thermal denaturation experiments conducted in bulk solution, as shown in Table 8. The results indicate that the opposite trend was observed with the biosensor with high oligonucleotide packing density.

TABLE 8
Observed Tₘ (° C.) values for Optical Fibre Biosensors
with Low, Medium and High Oligonucleotide Packing Densities Using
Hybridization Buffers of Various Ionic Strengths and d(A₉GA₁₀)-
fluorescein as the Complementary Material

| | Low | Medium | High |
|---|---|---|---|
| | Packing Density | Packing Density | Packing Density |
| [NaCl] (M) | Tₘ (° C.) | Tₘ (° C.) | Tₘ (° C.) |
| 0.3 | 39.2 ± 0.6 | 39.2 ± 0.6 | 31.1 ± 1.6 |
| 0.5 | 42.4 ± 0.5 | 42.0 ± 0.5 | 33.5 ± 1.0 |
| 1.0 | 48.5 ± 0.5 | 45.9 ± 0.5 | 36.5 ± 1.1 |
| ∂Tₘ/∂log [Na⁺] | 18 ± 2 | 12.7 ± 0.1 | 10.3 ± 0.2 |
| (° C.) | | | |

It may be that the higher packing density of immobilized DNA permits greater interaction between neighbouring strands under conditions of increased ionic strength within the hybridization solution and the nucleic acid membrane, resulting in greater destabilization of the hydrogen bonding accompanying hybridization and leading to greater deviations in the observed Tₘ in the higher ionic strength regions.

A comparison of the data shown in Table 6 and Table 7 with that shown in Table 4 shows that deviations in Tₘ observed as a result of the presence of a centrally located SBPM were significantly larger for experiments involving immobilized dsDNA relative to those observed for dsDNA floating freely in bulk solution. This observation is significant since it suggests that the thermodynamic selectivity of a hybridization assay using immobilized DNA may be significantly better than what may have otherwise been predicted by thermal denaturation experiments conducted in bulk solution. This enhancement of the deviation in the observed Tₘ values as a result of the presence of a centrally located SBPM suggests that hydrogen-bonding energetics associated with hybridization may be quite different in the interfacial environment than they may be in bulk solution. In order to examine the energetics of interfacial hybridization, the van't Hoff enthalpy changes and temperature-corrected standard enthalpy changes were computed for each of the denaturation experiments conducted here based on the method described elsewhere [28]. This model applies to denaturation occurring within a membrane of immobilized nucleic acids, with the complementary DNA freely able to float in and out of the membrane. The model assumes no interaction between neighbouring strands, and that the denaturation is a two-state transition. The van't Hoff enthalpy change is then given by the equation:

$$Δ {H}_{VH , {T}_{m}} = - [ ( \frac{1}{1 - {f}_{ss}} ) + ( \frac{1 - {f}_{ss min}}{{f}_{ss} - {f}_{ss min}} ) + {( \frac{{A}_{T}}{{B}_{T}} - 1 + \frac{{f}_{ss} - {f}_{ss min}}{1 - {f}_{ss min}} )}^{- 1} ] {{RT}_{m} ( \frac{\partial {f}_{ss}}{\partial T} )}_{T = {T}_{m}} \qquad ( 6 )$$

where ƒₛₛ is the total fraction of ssDNA present at any given time, ƒₛₛₘᵢₙ is the minimum ƒₛₛ possible in a system with a non-equal number of complementary strands, AT represents the total molar amount of complementary oligonucleotide and B_{T} represents the total molar amount of immobilized probe oligonucleotide. The value of fₛₛₘᵢₙ is then computed by the following equation:

$${f}_{ss min} = \frac{\vert {B}_{T} - {A}_{T} \vert}{{A}_{T} + {B}_{T}} \qquad ( 7 )$$

The van't Hoff enthalpy changes at Tₘ and the standard enthalpy changes corrected to a temperature of 40° C. for the different complementary oligonucleotides, oligonucleotide packing densities and hybridization buffer ionic strengths used in these experiments are summarized below in Table 9 and Table 10. Temperature corrections were made as described above according to the method of Breslauer [18]. The reference temperature used for all such corrections was chosen on the basis that it is operational temperature commonly used for hybridization assays conducted in our research group, chosen in order to enhance selectivity and hybridization kinetics.

The data shown in Table 9 and Table 10 show that the enthalpic change accompanying denaturation in an interfacial environment is significantly lower than that which is observed in experiments conducted in bulk solution, as shown Table 6. This suggests that there are significant differences in the nature of the hydrogen bonding involved with base pairing in an interfacial environment compared with that which occurs in bulk solution. There did not appear to be a relationship between the packing density of immobilized oligonucleotides and the reduction in the endothermicity of the denaturation. Thus, since observed Tₘ values are still of comparable magnitude as those which observed in experiments done in bulk solution, it is likely that there is a significant difference in entropy changes accompanying hybridization and denaturation in an interfacial environment, relative to those observed in experiments done in bulk solution. These differences in the entropy changes accompanying hybridization or denaturation may be dependent upon the density of immobilized oligonucleotides, as they may also be affected by the extent of interaction between neighbouring strands. Computation of entropy changes accompanying hybridization or denaturation in an interfacial environment would require computation of equilibrium constants for the hybridization process, which in turn requires knowledge of the ionic strength within the nucleic acid membrane [19]. Similar computations for processes occurring in bulk solution have been known to introduce significant error [18], and so these computations for immobilized nucleic acid systems will be left as future work.

It may be that interactions that are reducing the strength of the hydrogen bonding between base pairs are primarily between the immobilized oligonucleotides and the solid substrate surface. Salt present in the hybridization buffer can facilitate electrostatic interactions between the polyanionic phosphate backbone of the immobilized DNA and any charged functionalities present on the surface of the fused silica substrate. This interaction between immobilized strands and the surface of the solid substrate can restrict the changes in oligonucleotide secondary structure accompanying hybridization, which may alter the observed entropy change accompanying the hybridization or denaturation process. This interaction may also reduce the strength of hydrogen bonds formed between base pairs, which may be responsible for the reduction in the observed enthalpy changes accompanying the hybridization or denaturation process. Any structural restriction or reduction in the strength of the hydrogen bonding in interfacial nucleic acid hybrids may help contribute to the deviations in Tₘ reported above.

TABLE 9
van't Hoff and Standard Enthalpy Changes for Denaturation
of Immobilized Oligonucleotides with Different Packing Densities and
Ionic Strengths, using dA₂₀-Fluorescein (10⁻⁷ M)
as the Complementary Oligonucleotide.

| | Low | | Medium | | High | |
|---|---|---|---|---|---|---|
| | Packing Density | | Packing Density | | Packing Density | |
| | ΔH_{VH} | ΔH° | ΔH_{VH} | ΔH° | ΔH_{VH} | ΔH° |
| | (Tₘ) | (40° C.) | (Tₘ) | (40° C.) | (Tₘ) | (40° C.) |
| [NaCl] | (kcal/ | (kcal/ | (kcal/ | (kcal/ | (kcal/ | (kcal/ |
| (M) | mol) | mol) | mol) | mol) | mol) | mol) |
| 1.0 | 34 ± 2 | 41.5 | 30 ± 3 | 44.9 | 34.8 ± 3 | 48.0 |
| 0.5 | 37 ± 2 | 42.4 | 35 ± 3 | 44.1 | 35.4 ± 3 | 36.8 |
| 0.1 | 42 ± 3 | 41.7 | 43 ± 3 | 44.8 | 65.6 ± 3 | 49.4 |
| | Mean | 42 ± 1 | Mean | 45 ± 1 | Mean | 45 ± 7 |

TABLE 10
van't Hoff and Standard Enthalpy Changes for Denaturation of
Immobilized Oligonucleotides with Different Packing Densities and
Ionic Strengths, Using d(A₉GA₁₀)/d(A₉GA₁₀)-Fluorescein (10⁻⁷ M)
as the Complementary Oligonucleotide.

| | Low | | Medium | | High | |
|---|---|---|---|---|---|---|
| | Packing Density | | Packing Density | | Packing Density | |
| | ΔH_{VH} | ΔH° | ΔH_{VH} | ΔH° | ΔH_{VH} | ΔH° |
| | (Tₘ) | (40° C.) | (Tₘ) | (40° C.) | (Tₘ) | (40° C.) |
| [NaCl] | (kcal/ | (kcal/ | (kcal/ | (kcal/ | (kcal/ | (kcal/ |
| (M) | mol) | mol) | mol) | mol) | mol) | mol) |
| 1.0 | 50 ± 1 | 37.1 | 36 ± 4 | 39.9 | 38 ± 5 | 37.3 |
| 0.5 | 40 ± 1 | 36.4 | 34 ± 2 | 35.4 | 39 ± 5 | 37.8 |
| 0.3 | 36 ± 2 | 37.2 | 41 ± 4 | 40.5 | 39 ± 2 | 37.3 |
| | Mean | 37 ± 1 | Mean | 39 ± 3 | Mean | 37 ± 1 |

These data also suggest the magnitude van't Hoff enthalpy change accompanying denaturation in an interfacial environment does not display the same sensitivity to changes in ionic strength and, therefore, Tₘ, as was observed for experiments conducted in bulk solution. The sensitivities of ΔH_{VH} (Tₘ) to changes in Tₘ were a factor of 2-4 smaller for the transitions occurring at the interface of the optical biosensors relative to those observed for the experiments done in bulk solution, and were usually opposite in sign. This suggests that the changes in heat capacity that accompany the denaturation are not the same in an interfacial environment as they are in bulk solution, which may be due to local density changes in the membrane as a result of the denaturation. This further supports the notion that interfacial hybridization occurs in a physical environment that is significantly different than that of hybridization in bulk solution.

### Example 7

### Comparison of Nucleic Acid Hybridization in Interfacial Environments with Different Chemical Compositions: The Effects of Inclusion of Non-Nucleic Acid Oligomers at High Density

Experiments were done to examine the effects of inclusion of oligomers other than nucleic acids in immobilized films on the selectivity interfacial of nucleic acid hybridization. These experiments were done using a nucleic acid biosensor, based on total internal reflection fluorescence, wherein both probe oligonucleotides and ethylene glycol phosphate (EGp) oligomers were covalently immobilized in two different molar ratios to the surface of fused silica optical fibres via flexible polyether linkers. In these experiments, the method of immobilization used corresponded to that outlined in Example 4. Thermal denaturation profiles were obtained for such nucleic acid films in order to determine if trends with respect to interfacial hybridization using immobilized films comprised of nucleic acid conjugates only were in agreement with those observed in experiments using an immobilized film containing both nucleic acid conjugates and other species not expected to selectively bind nucleic acids. Since it has been shown that the efficiency of hybridization to nucleic acid films is dependent in part on the chemical nature of the interfacial environment [11, 12, 24], it is obviously important to examine the effects of film composition on the selectivity of interfacial nucleic acid hybridization.

Thermal denaturation experiments were done using fluorescein-labelled oligonucleotides that were fully complementary (dA₂₀-5′-fluorescein) or which contained a centrally-located SBPM (dA₉GA₁₀-5′-fluorescein) relative to the immobilized oligonucleotide probes (dT₂₀). In all experiments, the total target DNA concentration was 10⁻⁷ M. While the composition of the immobilized nucleic acid film was varied with respect to the relative amounts of DNA and oligo(EGp), analysis of immobilized species showed that the total density of immobilized molecules (both DNA and EGp) can be defined as being of high density. The effects of solution ionic strength were examined by varying the salt concentration of the phosphate-buffered saline in which the thermal denaturation experiments were done (0.1 M NaCl, 0.5 M NaCl or 1 M NaCl). Thermal denaturation of hybrids formed between the immobilized probes and fluorescein-labelled oligonucleotides was achieved by increasing the temperature of the hybridization solution within a range of 20-80° C., at a ramp-rate of 0.3° C. min⁻¹.

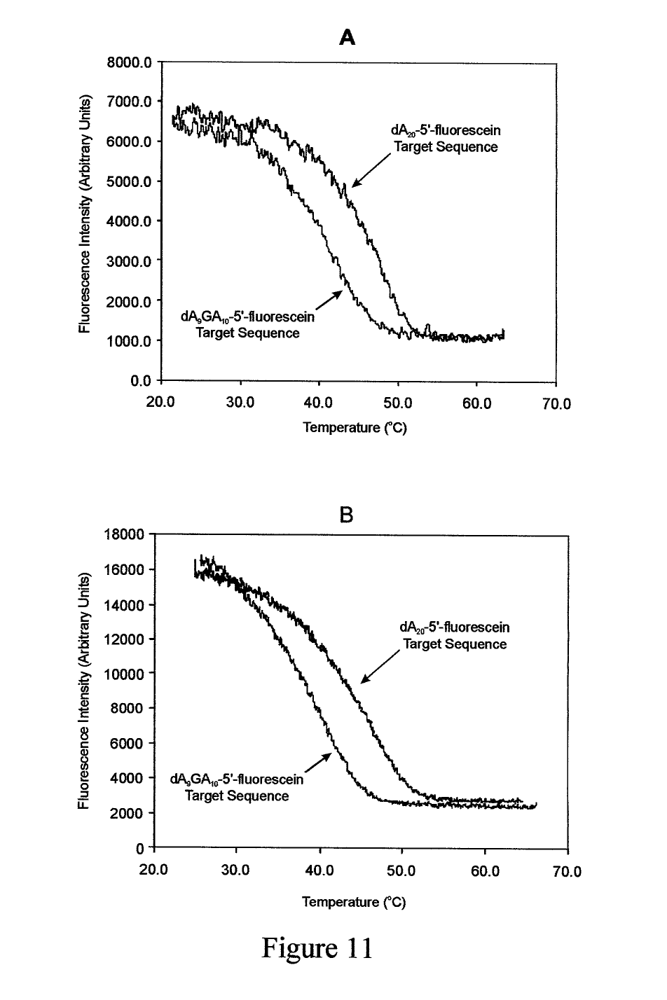

Examples of the raw data obtained from the thermal denaturation profiles of both fully complementary hybrids (dA₂₀-5′-fluorescein target in 0.5×PBS) and those containing a centrally located SBPM (dA₉GA₁₀-5′-fluorescein target in 0.5×PBS) measured using optical fibres functionalized with films containing dT₂₀ only and films containing dT₂₀ and oligo(EGp) in a 1:20 molar ratio are shown in FIG. 11. As described in Example 5, the thermal denaturation transition was assumed to have taken place as a two-state transition. It was assumed that full hybridization occurred and that complete denaturation was achieved in the high temperature regime. Normalization of all raw thermal denaturation data to yield the fraction of single-stranded DNA as a function of temperature was then done using baseline normalization methods analogous to those described in previous examples. Examples of normalized thermal denaturation profiles generated using optical fibres functionalized with films comprised of dT₂₀ and oligo(EGp) in a 1:20 molar ratio and hybridization buffers of various ionic strengths are shown in FIG. 12. The Tₘ data corresponding to sensors functionalized with films containing dT₂₀ only and those containing both dT₂₀ and oligo(EGp) in a 1:20 molar ratio are shown in Table 11.

TABLE 11
Thermal denaturation temperatures, Tₘ (° C.) for hybrids formed between dA₂₀-5'-
fluorescein (cDNA) or dA₉GA₁₀-5'-fluorescein (SBPM) and immobilized films comprised of
dT₂₀ only or dT₂₀ and oligo(EGp) in a 1:20 molar ratio in hybridization solutions of various ionic
strengths.

| | Immobilized | Immobilized | Immobilized | Immobilized | Immobilized | Immobilized |
|---|---|---|---|---|---|---|
| | dT₂₀ Only | dT₂₀ Only | dT₂₀ and | dT₂₀ and | dT₂₀ and | dT₂₀ and |
| | cDNA | SBPM | cDNA | oligo(EGp) | oligo(EGp) | oligo(EGp) |
| [NaCl] | Target | Target Tₘ | Target | cDNA | SBPM | cDNA |
| (M) | Tₘ (° C.) | (° C.) | ΔTₘ (° C.) | Target Tₘ (° C.) | Target Tₘ (° C.) | Target ΔTₘ (° C.) |
| 0.1 | 34.7 ± 0.7 | 27.6 ± 0.7 | 7 ± 1 | 30 ± 1 | 25.0 ± 0.6 | 5 ± 1 |
| 0.5 | 45.8 ± 0.6 | 40.3 ± 0.6 | 5.5 ± 0.8 | 44 ± 1 | 38 ± 1 | 6 ± 1 |
| 1 | 52.9 ± 0.4 | 45.6 ± 0.5 | 7.3 ± 0.6 | 48 ± 1 | 42 ± 1 | 6 ± 1 |

These data illustrate two important features. Firstly, the presence of another species (in this case, oligo{EGp}) appeared to exert a depressive effect on the observed Tₘ values relative to those observed using immobilized films comprised of dT₂₀ only. This further corroborates the notion that the interfacial environment of a nucleic acid hybrid has a direct effect on the thermodynamic stability of that hybrid. Furthermore, given that the total density of immobilized species is the same within experimental error, it is then likely that the effects of immobilization density and chemical environment of the interface both play a determining role in the thermodynamics of interfacial hybridization. Secondly, the differences in Tₘ between fully complementary hybrids and those containing a single base-pair mismatch for the two systems are in agreement with each other, and also display a similar trend to that outlined in Example 6, in that increasing the ionic strength of the hybridization solution did not result in a decrease in the Tₘ difference between fully complementary hybrids and those containing a centrally located SBPM. This is in contrast to conventionally accepted observations for experiments done in bulk solution and as described in Example 5.

These two features of the results presented in Table 11 have important consequences for hybridization assays that make use of interfacial nucleic acid hybridization. Firstly, it is possible to design an assay platform in which multiple sequences can be assayed simultaneously, as is the case in many of the commercially available DNA microarray platforms, such that the chemistry of immobilization of each probe sequence is designed to manipulate immobilization density and immobilization chemistry in order to tune the Tₘ of a particular sequence. By engineering an array of probe sequences, each with carefully designed immobilization density and chemistry, it is then be possible to generate an array of probe sequences with identical Tₘ values, regardless of the G-C content of the hybrids formed. This would allow the simultaneous assay of a number of different sequences at the same temperature with reduced loss of calibration. Secondly, the results of these experiments suggest that it is possible to control the relative selectivity of hybridization between a nucleic acid molecule and an immobilized probe relative to that of a nucleic acid molecule and its complementary molecule in solution. Engineering a nucleic acid film that (a) reduces the difference in Tₘ between a hybrid formed in an interfacial environment and that of a hybrid formed in bulk solution; and (b) maintains a larger difference in Tₘ between fully and partially complementary hybrids than that observed in bulk solution can provide a desirable product that can improve the selectivity and sensitivity of analytical methods based on interfacial nucleic acid hybridization.

It can also be observed in the data presented in FIG. 12 that the slope of the thermal denaturation profiles increase with increased ionic strength in the solution surrounding the sensors. This enhanced temperature sensitivity can provide for the development of analytical devices of extremely high selectivity. Based on these results, it is made obvious that devices containing immobilised films of high density nucleic acid, or mixed films of nucleic acids and oligomers, can be created and operated at temperature and solution ionic strength conditions such that only hybrids with fully complementary nucleic acids or nucleic acid analogues can be detected.

It will be appreciated that the above description relates to the preferred embodiments by way of example only. Many variations on the apparatus for delivering the invention will be obvious to those knowledgeable in the field, and such obvious variations are within the scope of the invention as described and claimed, whether or not expressly described.

All patents, patent applications, and publications referred to in this application are incorporated by reference in their entirety.

### REFERENCES

¹R. Blonder, E. Katz, Y. Cohen, N. Itzhak, A. Riklin, I. Willner, Anal. Chem., v. 68, p. 3151, 1996.

²R. Granzow and R. Reed, Biotechnology, v. 10, p. 390, 1992.

³B. König and M. Grätzel, Anal. Chim. Acta, v. 309, p. 19, 1995.

⁴K. M Millan, A. Saraullo, and S. M. Mikkelsen, Anal. Chem., v. 66, p. 2943, 1994.

⁵J. Wang, S. Bollo, J. L. Lopez Paz, E. Sahlin and B. Mukherjee, Anal. Chem., v. 71, p. 1910, 1999.

⁶H. Su, K. M. R. Kallury, M. Thompson and A. Roach, Anal. Chem., v. 66, p. 769, 1994.

⁷ F. Caruso, E. Rodda, D. N. Furlong, K. Niikura, and Y. Okahata, Anal. Chem., v. 69, p. 2043, 1997.

⁸A. P. Abel, M. G. Weller, G. L. Duveneck, M. Ehrat and H. M. Widmer, Anal. Chem., v. 68, p. 2905, 1996.

⁹P. A. E. Piunno, U. J. Krull, R. H. E. Hudson, M. J. Damha and H. Cohen, Anal. Chem., v. 67, p. 2635, 1995.

¹⁰H. Su, P. Williams and M. Thompson, Anal. Chem., v. 67 p. 1010, 1995.

¹¹T. M. Herne and M. J. Tarlov, J. Am. Chem. Soc., v. 119, p. 8916, 1997.

¹²R. Levicky, T. M. Herne, M. J. Tarlov, and S. K. Satija, J. Am. Chem. Soc., v. 120, p. 9787, 1998.

¹³M. -T. Charreyre, O. Tcherkassaya, et al. Langmuir, v. 13, p. 3103, 1997.

¹⁴A. V. Fotin, A. L. Drobyshev, D. Y. Proudnikov, A. N. Perov, A. D. Mirzabekov, Nuc. Ac. Res., v. 26, p. 1515, 1998.

¹⁵M. S. Shchepinov, S. C. Case-Green, and E. M. Southern, Nuc. Ac. Res., v. 25, p. 1155, 1997.

¹⁶E. Marshall, Science, v.268, p.1270, 1995.

¹⁷M. Schena, D. Shalow. R. Heller, A. Chai, P. O. Brown, R. W. Davis, Proc. Nat'l. Acad. Sci. USA, v.93, p.10614, 1996.

¹⁸“Recent Advances in Environmental Chemical Sensors and Biosensors”, ACS Symposium Series, in press

¹⁹M. Thompson and L. M. Furtado, Analyst, v. 124, p. 1133, 1999.

²⁰Affymetrix, Inc., 3380 Central Expressway, Santa Clara, Calif., USA.

²¹ S. P. A. Fodor, J. L. Read, M. C. Pirrung, L. Stryer, A. Tsai Lu, D. Solas, Science, v.251, p.767, 1991.

²²M. Chee, R. Yang, E. Hubbell, A. Berno, X. C. Huang, D. Stem, J. Winkler, D. J. Lockhart, M. S. Morris, S. P. A Fodor, Science, v.274, p. 610, 1996.

²³(i) Nanogen, Inc., 10398 Pacific Center Court, San Diego, Calif., USA, 27, (ii) R. G. Sosnowski, E. Tu, W. F. Butler, J. P. O'Connell, M. J. Heller, Proc. Natl. Acad. Sci., v. 94, pp. 1119-1123, 1997.

²⁴U. Maskos and E. M. Southern, Nuc. Acids Res., v. 20, p. 1679, 1992.

²⁵T. V. Chalikian, J. Völker, G. E. Plum, and K. J. Breslauer, Proc. Nat'l. Acad. Sci. USA, v. 96, p.7853, 1999.

²⁶K. J. Bresaluer in “Methods in Molecular Biology, Vol. 26: Protocols for Oligonucleotide Conjugates”, S. Agrawal, Ed., p. 347, Humana Press, NJ, 1994.

²⁷R. A. Alberty and R. J. Silbey, Physical Chemistry, J. Wiley & Sons, 1st ed., 1992.

²⁸P. A. E. Piunno, J. H. Watterson, C. C. Wust, and U. J. Krull, Anal. Chim. Acta v. 400, p. 73, 1999.

We claim:

1. A substrate for hybridization comprising a plurality of first nucleic acid alone or in combination with a plurality of one or more oligomers that are not nucleic acids immobilized on at least a portion of the substrate in a medium-high or high immobilization density

2. The substrate of claim 1 wherein a second nucleic acid having a region of contiguous nucleotides that are complementary to all or part of at least one of the first nucleic acids will selectively hybridize to the at least one first nucleic acid.

3. The substrate of claim 2 wherein, in an assay, the difference in Tₘ between
(i) a fully-matched complex immobilized to the substrate, the complex comprising the first nucleic acid and the second nucleic acid; and
(ii) a mismatched complex immobilized to the substrate, the complex comprising the first nucleic acid and a second nucleic acid having a single nucleotide mismatch;
is not decreased compared to the difference in Tₘ between the complexes in low immobilization density.

4. The substrate of claim 3 wherein difference in Tₘ between (i) and (ii) is increased compared to the difference in Tₘ between the complexes in low immobilization density.

5. The substrate of claim 4 wherein the difference in Tₘ is at least 5 degrees Celsius.

6. The substrate of any of claims 1 to 5, wherein the medium-high immobilization density comprises oligomers on the substrate so that the ratio (rₛ) of the mean centre-to-centre separation distance of the oligomers to the average length of immobilized oligomers is less than or equal to 2.

7. The substrate of claim 1 wherein the high immobilization density comprises oligomers on the substrate so that the ratio (rₛ) of the mean centre-to-centre separation distance of the oligomers to the average length of immobilized oligomers less than or equal to 1.7

8. The substrate of claim 1 wherein the nucleic acid is a dendritic assembly containing nucleic acid residues.

9. The substrate of claim 1 wherein the first nucleic acids are immobilized to the substrate by a linker.

10. The substrate of claim 9 wherein the linker comprises a polyether moiety, a poly(ethylene oxide) moiety or a polymeric moiety.

11. The substrate of claim 1 wherein the one or more oligomers other than nucleic acids are immobilized to the substrate by a linker.

12. The substrate of claim 11 wherein the linker comprises a polyether moiety, a poly(ethylene oxide) moiety or a polymeric moiety.

13. The substrate of claim 1 wherein the first nucleic acids comprise identical nucleic acid sequence.

14. The substrate of claim 1 wherein the first nucleic acids comprise a mixture of nucleic acid sequences.

15. The substrate of claim 1 wherein the first nucleic acids comprise a mixture of nucleic acid sequences and/or nucleic acid analogues and/or nucleotide analogue sequences.

16. A substrate of claim 1 wherein a plurality of first nucleic acids and a plurality of one or more oligomers that are not nucleic acids are immobilized on the substrate.

17. The substrate of claim 1, wherein the one or more oligomers comprise polyelectrolyte moieties and/or polymeric moieties.

18. The substrate of claim 1 wherein the one or more oligomers are polyethers.

19. The substrate of claim 1 wherein the second nucleic acid and the at least one first nucleic acid hybridize in a high ionic strength solution.

20. The substrate of claim 18 wherein the high ionic strength solution is at least 0.3 mol/L.

21. The substrate of claim 1 wherein the interfacial hybridization for fully complementary nucleic acids exhibits enhanced sensitivity to temperature

22. The substrate of claim 1 wherein the substrate comprises an optical fiber, an optical wave-guide, a spot on a microarray chip, a microtiter plate well, a metal film for surface plasmon resonance determination, a glass bead, a planar waveguide, a quartz oscillator, a ceramic oscillator, a conductive electrode material, a semi-conductive electrode material, a plastic sample compartment, an optical component or a pyroelectric material.

23. The substrate of claim 1 which is a substrate for a hybridization assay.

24. The substrate of claim 1 further comprising a plurality of first nucleic acids alone or in combination with a plurality of one or more oligomers that are not nucleic acids immobilized on at least a portion of the substrate in a low immobilization density.

25. A method of preparing a substrate for hybridization, comprising immobilizing a plurality of first nucleic acids alone or in combination with one or more oligomers that are not nucleic acids to the substrate in a medium-high or high immobilization density.

26. The method of preparing a substrate for hybridization of claim 25 comprising immobilizing a plurality of first nucleic acids to the substrate alone or in combination with one or more oligomers that are not nucleic acids in a high immobilization density.

27. The method of claim 25 wherein the nucleic acids are a dendritic assembly containing nucleic acid residues.

28. The method of claim 25 wherein the first nucleic acids are connected to the substrate by a linker.

29. The method of claim 28 wherein the linker comprises a polyether moiety, a poly(ethylene oxide) moiety or a oligomer moiety.

30. The method of claim 25 wherein the first nucleic acids comprise an identical nucleic acid sequence.

31. The method of claim 25 wherein the first nucleic acids comprise a mixture of nucleic acid sequences.

32. The method of claim 25 wherein the substrate comprises an optical fiber, an optical wave-guide, a spot on a microarray chip, a microtiter plate well, a metal film for surface plasmon resonance determination, a planar waveguide, a quartz oscillator, a ceramic oscillator, a conductive electrode material, a semi-conductive electrode material, a glass bead, a plastic sample compartment, an optical component or a pyroelectric material.

33. A method of hybridizing nucleic acids comprising:
providing a substrate including a plurality of first nucleic acids or first nucleic acids and oligomers which are not nucleic acids on the substrate, having a medium-high or high immobilization density; and
contacting the substrate with at least one second nucleic acid having a region of contiguous nucleotides that are complementary to all or part at least one of the first nucleic acids, so that the second nucleic acid hybridizes to the at least one first nucleic acid.

34. The method of claim 33, wherein the second nucleic acid selectively hybridizes to the at least one first nucleic acid.

35. The method of claim 33 wherein, in an assay, the difference in Tₘ between
(i) a fully-matched complex immobilized to a substrate, the complex comprising the first nucleic acid and the second nucleic acid; and
(ii) a mismatch complex immobilized to a substrate, the complex comprising the first nucleic acid and a second nucleic acid having a single nucleotide mismatch; is increased or maintained relative to the difference in Tₘ between the complexes in low immobilization density.

36. The method of claim 35, wherein the difference in Tₘ is at least 5 degrees Celsius.

37. The method any of claims 33 wherein the second nucleic acid and the at least one first nucleic acid hybridize in a high ionic strength solution.

38. The method of claim 37 wherein the high ionic strength solution is at least 0.3 mol/L.

39. The method of claim 33 wherein the hybridization comprises a Tₘ inversion effect.

40. The method of claim 33 wherein the hybridization for fully complementary nucleic acids exhibits enhanced sensitivity to temperature

41. The method of claim 33 wherein the first nucleic acids comprise an identical nucleic acid sequence.

42. The method of claim 33 wherein the first nucleic acids comprise a mixture of nucleic acid sequences.

43. The method of claim 33 wherein the first nucleic acids comprise a mixture of nucleic acid sequences.

44. The method of claim 33 wherein the substrate comprises an optical fiber, an optical waveguide, a spot on a microarray chip, a microtiter plate well, a metal film for surface plasmon resonance determination, a planar waveguide, a quartz oscillator, a ceramic oscillator, a conductive electrode material, a semi-conductive electrode material, a glass bead, a plastic sample compartment, an optical component or a pyroelectric material.

45. The method of claim 33 wherein the substrate is contacted with a mixture of nucleic acids including the at least one second nucleic acid.

46. The method of claim 33 further comprising a step of detecting hybridization.

47. The method of claim 46 wherein hybridization is detected by detection of fluorescence.

48. A method of detecting the presence of a genetic target in a test sample, comprising:
providing a substrate including a plurality of genetic marker nucleic acids immobilized to the substrate, alone or in combination with one or more oligomers at a medium-high or high immobilization density;
contacting the substrate with a test sample comprising a mixture of nucleic acids so that a second nucleic acid having a region of contiguous nucleotides that are complementary to all or part of at least one of the genetic marker nucleic acids hybridizes to at least one first nucleic acid; and
detecting hybridization of the genetic marker to the second nucleic acid, wherein hybridization is indicative of the presence of a genetic target in the sample.

49. The method of claim 48, wherein the second nucleic acid selectively hybridizes to the at least one genetic marker nucleic acid.

50. The method of claim 48 wherein, in an assay, the difference in Tₘ between
(i) a fully-matched complex immobilized to a substrate, the complex comprising the first nucleic acid and the second nucleic acid; and
(ii) a mismatch complex immobilized to a substrate, the complex comprising the first nucleic acid and a second nucleic acid having a single nucleotide mismatch;
is increased or maintained relative to the difference in Tₘ between the complexes in low immobilization density.

51. The method of claim 48 wherein the difference in Tₘ is at least 5 degrees Celsius.

52. The method of any of claims 47-50 wherein the hybridization comprises a Tₘ inversion effect.

53. The method of claim 48 wherein the second nucleic acid and the at least one first nucleic acid hybridize in a high ionic strength solution.

54. The method of claim 53 wherein the high ionic strength solution is at least 0.3 mol/L.

55. The method of claim 48 wherein the hybridization for fully complementary nucleic acids exhibits enhanced sensitivity to temperature

56. The method of claim 48 wherein the first nucleic acids comprise an identical nucleic acid sequence.

57. The method of claim 48 wherein the first nucleic acids comprise a mixture of nucleic acid sequences.

58. The method of claim 48 wherein the first nucleic acids comprise a mixture of nucleic acid sequences.

59. The method of claim 48 wherein the substrate comprises an optical fiber, an optical waveguide, a spot on a microarray chip, a microtiter plate well, a metal film for surface plasmon resonance determination a planar waveguide, a quartz oscillator, a ceramic oscillator, a conductive electrode material, a semi-conductive electrode material, a glass bead, a plastic sample compartment, an optical component or a pyroelectric material.

60. The method of claim 48 wherein the genetic target comprises a disease marker nucleic acid and wherein hybridization is indicative of the presence of a disease state in the subject sample.

61. The method of claim 48 wherein the test sample comprises a sample obtained from a patient or derived from nucleic acids obtained from a patient.

62. The method of claim 48 wherein the nucleic acids are derived by a nucleic acid amplification method.

63. The method of claim 48 wherein the genetic target comprises an environmental marker nucleic acid, a food marker nucleic acid or a biowarfare agent nucleic acid, and wherein hybridization is indicative of the presence of the genetic target in the sample.

64. The method of claim 48 wherein the test sample comprises a sample obtained from an environmental source, food source, patient source or derived from one of the aforementioned sources.

65. The method of claim 48 wherein the nucleic acids to be tested are derived by a nucleic acid amplification method.

66. The method of claim 48 wherein hybridization of the marker to the second nucleic acid is detected with an indicator agent that indicates hybridization of the marker to the second nucleic molecule.

67. The method of claim 48 wherein the nucleic acids to be tested comprise an indicator agent.

68. The method of claim 67 wherein the indicator agent comprises a fluorophore.

69. The method of claim 33 wherein the hybridization is conducted below the Tₘ of a complex of the first nucleic acid and second nucleic acid but above the Tₘ of a complex of the a first nucleic acid and a complementary nucleic acid having a single nucleotide mismatch.

70. The method of claim 48 wherein the hybridization is conducted below the Tₘ of a complex of the first nucleic acid and second nucleic acid but above the Tₘ of a complex of the a first nucleic acid and a complementary nucleic acid having a single nucleotide mismatch.

71. The use of the substrate of claim 1 for diagnosing a disease state or detecting a genetic target.

72. A kit for detecting the presence of a genetic target in a test sample, comprising one or more substrates of any of claim 1.

73. The kit of claim 72 further comprising a hybridization buffer.

74. The kit of claim 72 wherein the genetic target comprises a disease marker nucleic acid, an environmental marker nucleic acid, a food marker nucleic acid or a biowarfare agent nucleic acid.

75. A method for identifying or isolating a target nucleic acid from a mixture containing nucleic acids which comprises the steps of:
providing a substrate of claim 1 wherein the first nucleic acids comprise a sequence that is complementary at least in part to the target nucleic acid; and
contacting the substrate with the mixture containing nucleic acids such that any target nucleic acid present in the mixture can hybridize to the first nucleic acids on the substrate.

76. The method of claim 75 wherein the step of contacting the substrate with the mixture is performed at high ionic strength.

77. The method of claim 75 wherein the mixture containing nucleic acids can contain nucleic acids that differ from the target nucleic acid by a single base change.
